(19) United States
(12) Patent Application Publication
(10) Pub. No.: US 2025/0276116 A1
(43) Pub. Date: Sep. 4, 2025

(54) METHODS OF MITIGATING MYOCARDIAL DAMAGE DUE TO EXTRACORPOREAL MEMBRANE OXYGENATION

(71) Applicant: Tufts Medical Center, Inc., Boston, MA (US)

(72) Inventor: Navin K. KAPUR, Hanover, MA (US)

(21) Appl. No.: 18/273,121

(22) PCT Filed: Jan. 21, 2022
(86) PCT No.: PCT/US2022/013394

§ 371 Date: Jul. 19, 2023

Related U.S. Application Data
(60) Provisional application No. 63/140,651, filed on Jan. 22, 2021.

(51) Int. Cl. A61M 1/16; A61K 31/185; A61K 31/445; A61K 38/07; A61M 60/109; A61M 60/13; A61M 60/216; A61M 60/38; A61M 60/411; A61P 9/10

(52) U.S. Cl. CPC A61M 1/1698; A61K 31/185; A61K 31/445; A61K 38/07; A61M 60/109; A61M 60/13; A61M 60/216; A61M 60/38; A61M 60/411; A61P 9/10

(57) ABSTRACT

The invention provides methods, compositions, systems, and kits for use in reducing or preventing myocardial damage due to extracorporeal membrane oxygenation.

### SEQUENCE LISTING

The instant application contains a Sequence Listing which has been submitted electronically in ASCII format and is hereby incorporated by reference in its entirety. Said ASCII copy, created on Jan. 19, 2022, is named 00398-561WO2_Sequence_Listing_1_18_22_ST25 and is 15,663 bytes in size.

### FIELD OF THE INVENTION

This invention is in the field of cardiopulmonary medicine.

### BACKGROUND OF THE INVENTION

Nearly 23 million people suffer from heart failure (HF) worldwide. HF affects approximately 7 million individuals in the United States alone, with healthcare expenditures reaching nearly 40 billion dollars per year (Savarese et al., Card. Fail. Rev. 3(1):7-11, 2017; Heidenreich, Circ. Heart Failure 6(3):606-619, 2013). Acute myocardial infarction (AMI) is a leading cause of HF and occurs in over 650,000 individuals per year in the United States (Benjamin et al., Circulation 139(10):e56-e528, 2019). Despite early revascularization in AMI, recent reports suggest that for every 5% increase in infarct size, one-year mortality and HF hospitalization are increased by 20% (Stone et al., J. Am Coll. Cardiol. 67(14):1674-1683, 2016). Paradoxically, coronary reperfusion itself may accelerate myocardial injury and cardiomyocyte death in AMI (Hausenloy et al., J. Clin. Invest. 123(1):92-100, 2013; Heusch, Circ. Res. 120(9):1477-1486, 2017). Despite improvements in reperfusion for AMI, subsequent HF remains a significant healthcare problem and new approaches to reduce myocardial damage in AMI are needed.

Prior attempts to limit infarct size using pharmacologic agents targeting reperfusion injury have failed to show clear clinical benefit (Kapur et al., JACC Heart Fail. 3(11):873-882, 2015). Due to the mandate for rapid reperfusion, this failure may reflect the inability of drugs to reach the area at risk and limited time for any therapeutic effect before reperfusion. Delaying reperfusion to deliver a cardioprotective drug may cause harm by increasing the duration of myocardial ischemia and subsequent circulatory collapse. New and improved strategies to optimize cardioprotective drug delivery before opening a blocked artery while providing systemic circulatory support in AMI are needed.

Use of veno-arterial extracorporeal membrane oxygenation (VA-ECMO) has grown exponentially over the last decade with a more than 11-fold increase for AMI patients in the United States and Europe (Vallabhajosyula et al., Circ. Heart Fail. 12(12):e005929, 2019; Becher et al., Circ. 138(20):2298-2300, 2018). VA-ECMO oxygenates and displaces venous blood into the arterial system using peripherally implanted venous drainage and arterial return cannulas, an oxygenator, and centrifugal flow pump. VA-ECMO provides full circulatory and respiratory support, is simple to implant, is widely available, and is therefore being used more commonly in AMI, shock, and high-risk coronary intervention (van den Brink et al., Neth. Heart J. 26(2):76-84, 2018; van den Brink et al., Neth. Heart J. 28(3):139-144, 2020; Sheu et al., Crit. Care Med. 38(9):1810-1817, 2010). Despite growing use, in-hospital mortality among VA-ECMO recipients remains high and is worse among AMI versus non-AMI recipients (Acharya et al., J. Am. Coll. Cardiol. 7(8):1001-1002, 2020; Vallabhajosyula et al., Circ. Heart Fail. 12(12):e005929, 2019). In-hospital mortality averages around 40% and survival without need for heart transplantation or a durable ventricular assist device is low (Garan et al., J. Am. Heart Assoc. 8(9):e01217122-23, 2019; Dangers et al., Crit Care Med. 45(8):1359-1366, 2017).

There is a need for new approaches to limiting myocardial damage due to AMI and VA-ECMO treatment.

### SUMMARY OF THE INVENTION

The invention provides methods for reducing or preventing myocardial damage in a subject caused by extracorporeal membrane oxygenation (ECMO), the methods comprising administering a cardioprotective agent to the subject and treating the subject with ECMO.

The invention yet further provides methods of reducing or preventing myocardial damage in a subject caused by ECMO, the methods comprising treating the subject with normoxemic ECMO.

The invention still further provides methods for reducing or preventing myocardial damage in a subject caused by ECMO, the method comprising treating the subject with left ventricular (LV) decompression prior to ECMO treatment.

In one aspect of the methods, (i) the myocardial damage is caused by reduction or depletion of tafazzin levels in cardiac muscle cells of the subject, and the methods restore or maintain said tafazzin levels, (ii) the myocardial damage is caused by reduction or depletion of cardiolipin levels in cardiac muscle cells of the subject, and the method restores or maintains said cardiolipin levels, (iii) the myocardial damage is caused by increased iAAA protease (e.g., YME1) activity in cardiac muscle cells of the subject, and the method decreases iAAA protease levels, in order to restore or maintain tafazzin levels; and/or (iv) the mitochondrial protective agent stabilizes cardiolipin, protects tafazzin from ECMO-induced depletion or reduction, or targets a protease that causes tafazzin degradation.

The invention yet further provides methods of reducing, preventing, or treating myocardial damage in a subject caused by ECMO, the methods comprising restoring or maintaining tafazzin levels in cardiac muscle cells of the subject by enzyme replacement, gene therapy, CRISPR, mRNA therapy, protease inhibitors, or siRNA, which is directed to tafazzin directly, cardiolipin, and/or an iAAA protease (e.g., YME1).

In one aspect of the methods, the methods further comprise treating the subject with ECMO, optionally with preemptive LV decompression, which optionally is carried out prior to initiation of ECMO.

In another aspect of the methods, the methods further comprise administering a cardioprotective agent to the subject.

In another aspect of the methods, the cardioprotective agent is administered before or concurrently with the ECMO treatment.

In another aspect of the methods, the cardioprotective agent is administered before, during, or after ECMO treatment, or in any combination thereof.

In another aspect of the methods, the subject is being treated for ischemic heart disease.

In another aspect of the methods, the ischemic heart disease is selected from acute myocardial infarction, heart failure, shock, and high risk percutaneous coronary intervention (PCI).

In another aspect of the methods, the methods further comprise treating the subject with coronary artery reperfusion.

In another aspect of the methods, in instances of treatment with a cardioprotective agent, the cardioprotective agent is administered before the start of coronary artery reperfusion.

In another aspect of the methods, the cardioprotective agent is administered at least 30 minutes before the start of coronary artery reperfusion.

In another aspect of the methods, the myocardial damage comprises a myocardial infarction or an increase in the size of an already existing myocardial infarction.

In another aspect of the methods, the myocardial damage comprises left ventricular (LV) injury.

In another aspect of the methods, the myocardial damage is characterized by oxidative stress.’

In another aspect of the methods, the myocardial damage is ischemia-reperfusion injury.

In another aspect of the methods, the subject has or is at risk of developing myocardial infarction, heart failure, cardiac arrest, heart muscle disease, myocarditis, sepsis, hypothermia, post-transplant complications, cardiogenic shock, cardio-respiratory failure, respiratory failure, lung infection, acute respiratory distress syndrome, pulmonary embolism, congenital diaphragmatic hernia, influenza, pulmonary hypertension, pneumonia, respiratory failure, trauma, or Covid-19, or is subject to treatment with or by a ventricular assist device, heart transplant, lung transplant, heart surgery, or cardiac catheterization.

In another aspect of the methods, in instances of treatment with a cardioprotective agent, the cardioprotective agent is administered by an intra-arterial, intra-coronary, intra-myocardial, intra-epicardial, pericardial, or intravenous route, or via the ECMO circuit.

In another aspect of the methods, the methods further comprise intravenous administration of a cardioprotective agent.

In another aspect of the methods, in instances of treatment with coronary artery reperfusion, ECMO treatment is maintained during the coronary artery reperfusion.

In another aspect of the methods, the ECMO is veno-arterial ECMO or veno-venous ECMO.

In another aspect of the methods, the VA-ECMO is peripheral VA-ECMO.

In another aspect of the methods, the ECMO is normoxemic ECMO.

In another aspect of the methods, the methods further comprise LV decompression before initiation of ECMO.

In another aspect of the methods, the LV decompression is carried out at least about 30 minutes before initiation of ECMO.

In another aspect of the methods, the LV decompression is carried out using a trans-valvular pump (TVP), an intra-aortic balloon pump (IABP), trans-aortic drainage catheters, left atrial decompression, pulmonary artery drainage cannulas, or placement of an arterial ECMO cannula in the thoracic aorta.

In another aspect of the methods, in instances of treatment with a cardioprotective agent, the cardioprotective agent comprises a mitochondrial protective agent, an antioxidant, or an oxygen radical scavenger wherein the oxygen radical scavenger is optionally selected from a nitroxide, such as Tempol (4-hydroxy-2,2,6,6-tetramethylpiperydine-1-oxyl) or Tiron (4,5-dihydroxy-1,3-benzenedisulfonic acid).

In another aspect of the methods, the mitochondrial protective agent stabilizes cardiolipin, protects tafazzin from ECMO-induced depletion or reduction, or targets a protease that causes tafazzin degradation, wherein optionally a mitochondrial protease inhibitor is used, which optionally is a ClpP inhibitor (e.g., AV167, TG42, TG53, and TG54) or a ClpXP inhibitor.

In another aspect of the methods, in instances of treatment with a cardioprotective agent, the cardioprotective agent comprises an aromatic tetrapeptide.

In another aspect of the methods, in instances of treatment with a cardioprotective agent, the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

In some embodiments, the LV decompression is commenced at least 10, 15, 20, 25, 30, 35, 40, 45, 50, 55, 60, 75, 90, 105, 120, 135, 150, 165, 180, 195, or 210 minutes before ECMO treatment begins.

In some embodiments, the LV decompression is carried out no longer than 90, 105, 120, 135, 150, 165, 180, 195, or 210 minutes before ECMO treatment is commenced.

In some embodiments, the LV decompression commences 10-180, 15-150, 20-120, 25-90, 15-45, 30-60, 45-75, 60-90, 75-105, 90-120, 105-135, 120-150, 135-165, 150-210, 105-195, or 120-180 minutes before commencement of ECMO treatment.

In some embodiments, the flow rate of LV decompression is greater than the flow rate of ECMO.

In some embodiments, the flow rate for LV decompression is 3-5 L/minute, while the flow rate for ECMO is 2-4 L/minute, provided that the LV decompression flow rate is greater than the ECMO flow rate.

In some embodiments, the LV decompression flow rate is 3.5 to 4.5 L/minute, while ECMO flow rate is 3-4 L/minute, provided that the LV decompression flow rate is greater than the ECMO flow rate.

In some embodiments, the LV decompression flow rate is 3.5 L/minute, while the ECMO flow rate is 4 L/minute.

In some embodiments, once ECMO has begun, combined LV decompression and ECMO treatment is carried out for at least 30, 45, 60, 75, 90, 105, 120, 135, 150, 165, 180, 195, or 210 minutes.

In some embodiments, the combined LV decompression and ECMO treatment is carried out for no more than 90, 105, 120, 135, 150, 165, 180, 195, or 210 minutes.

In some embodiments, the combined LV decompression and ECMO treatment is carried out for 30-60, 45-75, 60-90, 75-105, 90-120, 105-135, 120-150, 135-165, 150-180, 165-195, or 180-210 minutes.

The invention yet further provides systems comprising an ECMO machine and a cardioprotective agent or an ECMO machine and a LV decompression device.

In one aspect of the system, the cardioprotective agent comprises a mitochondrial protective agent, an antioxidant, or an oxygen radical scavenger.

In another aspect of the systems, the mitochondrial protective agent stabilizes cardiolipin, protects tafazzin from ECMO-induced depletion or reduction, or targets a protease that causes tafazzin degradation.

In another aspect of the systems, the cardioprotective agent comprises an aromatic tetrapeptide.

In another aspect of the systems, the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

The invention yet further provides compositions for use in reducing or preventing myocardial damage in a subject caused by extracorporeal membrane oxygenation (ECMO), the compositions comprising a cardioprotective agent.

In one aspect of the compositions for use, the cardioprotective agent comprises a mitochondrial protective agent, an antioxidant, or an oxygen radical scavenger.

In another aspect of the compositions for use, the mitochondrial protective agent stabilizes cardiolipin, protects tafazzin from ECMO-induced depletion or reduction, or targets a protease that causes tafazzin degradation.

In another aspect of the compositions for use, the cardioprotective agent comprises an aromatic tetrapeptide.

In another aspect of the compositions for use, the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

The invention yet further provides kits comprising: (a) one or more disposable medical product for use with an ECMO machine, and (b) a cardioprotective agent.

In one aspect of the kits, the one or more disposable medical product is selected from the group consisting of a connector, a cannula, a tubing, or a filter.

In one aspect of the kits, the cardioprotective agent comprises a mitochondrial protective agent, an antioxidant, or an oxygen radical scavenger.

In one aspect of the kits, the mitochondrial protective agent stabilizes cardiolipin, protects tafazzin from ECMO-induced depletion or reduction, or targets a protease that causes tafazzin degradation.

In one aspect of the kits, the cardioprotective agent comprises an aromatic tetrapeptide.

In one aspect of the kits, the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

Other features and advantages of the invention will be apparent from the following description and the claims.

### BRIEF DESCRIPTION OF THE DRAWINGS

FIG. 1A illustrates how VA-ECMO displaces and oxygenates venous blood into the arterial system. FIGS. 1B and 1C show that VA-ECMO activation decreases LV stroke work (area within the PV loop) and increases LV pressure and volume (PVA). FIG. 1D illustrates VA-ECMO combined with a trans-valvular pump (TVP). FIGS. 1E and 1F show that VA-ECMO plus TVP activation maximally reduces LV pressure and volume and PVA. FIG. 1G shows that LV pressure decreases with VA-ECMO+TV-Pump. FIG. 1H shows that, compared to IRI, VA-ECMO increases infarct size normalized to the area at risk (IS/AAR). TV-pump activation before (Pre-TVP), but not after (Post-TVP) VA-ECMO initiation attenuates infarct expansion with VA-ECMO (n=4/group).

FIG. 2A shows infarct sizes with ischemia-reperfusion (IRI), VA-ECMO with hyperoxemic PaO2 levels, and VA-ECMO with normoxemic (N) PaO2 levels. FIG. 2B is shows that infarct size correlates directly with PaO2 levels on ECMO. FIG. 2C shows that N-ECMO increases the collateral flow index (CFI) before coronary reperfusion. FIG. 2D shows wave intensity analysis, which shows that VA-ECMO activation attenuates the coronary backward expansion wave (BEW) in swine models of AMI. p<0.05 vs *IRI or † VA-ECMO.

FIGS. 3A-3B show LV levels of tafazzin (TAZ) and cardiolipin (CL) in healthy swine with and without 6 hours of VA-ECMO. FIGS. 3C-3D show human LV TAZ levels with and without VA-ECMO. FIG. 3E shows western blot analysis of mitochondrial tafazzin levels with positive and negative controls from tafazzin knockout (KO) mice and recombinant tafazzin. FIG. 3F shows mitochondrial tafazzin, total CL, and MLCL:CL levels within the area at risk. FIG. 3G shows electron transport chain (ETC) complex function relative to sham. FIG. 3H shows hydrogen peroxide (H202) and peroxidized lipid levels within the infarct zone. FIGS. 3I and 3J show BCL-2/BAX, BCL-XL, cleaved Caspase-3, LC3, and p62 levels within the infarct zone (n=3-4/group). FIG. 3K shows Ymel1 protein levels in the infarct zone. FIG. 3L shows rTAZ and FIG. 3M shows TAZ levels after treatment with Ymel1 in vitro and in isolated mitochondria. FIGS. 3N-3P shows confocal microscopy quantifying CM mitochondria and lysosome levels in the infarct zone.

FIG. 4A shows graphs of infarct sizes with elamipretide and/or VA-ECMO. FIG. 4B shows quantification of total CL and MLCL:CL ratios, and FIG. 4C shows mitochondrial protease activity and H202 levels in the infarct zone. FIG. 4D shows TTC staining for infarct size (white area) and the area at risk (dark area). FIG. 4E shows ETC complex function with elamipretide and VA-ECMO (n=3-4/group).

FIG. 5A shows LV scar images with reperfusion (IRI) or VA-ECMO. FIG. 5B shows quantified LV scar total LV, ejection fraction, and stroke volume. FIG. 5C shows BNP levels after AMI. FIG. 5D shows ETC complex function in the infarct zone 28 days after AMI (n=3-4/group).

FIG. 6 shows that the combination of a cardioprotective stabilizing agent (i.e., elamipretide) given before VA-ECMO can limit LV scar size and late-term LV injury.

FIG. 7 shows the effect of device configurations on left ventricular pressure volume area (PVA)-a well-established surrogate for myocardial oxygen consumption. Bailout ECPella (ECMO then Impella) does not significantly reduce PVA compared to ECMO alone. Pre-emptive ECPella (Impella, then 30 minutes of delay before ECMO) significantly reduces PVA compared to ECMO alone or Bailout ECPella.

FIG. 8 shows that both EC-Pella configurations decreased infarct size (FIG. 8A), increased cardioprotective signaling via the reperfusion injury salvage kinase pathway (FIG. 8B), reduced apoptosis and pro-apoptotic signaling (FIG. 8C).

FIG. 9 shows that pre-emptive, but not bailout, ECPella preserved mitochondrial preserved mitochondrial tafazzin levels (FIGS. 9A-9B), increased mitochondrial cardiolipin levels (FIG. 9C), and reduced the ratio of immature: mature cardiolipin (MLCL:CL ratio; FIG. 9D) within the infarct zone compared to ECMO alone. Pre-emptive ECPella also increased mitochondrial Complex I activity compared to ECMO or reperfusion alone, but Bailout ECPella did not (FIG. 9E).

### DETAILED DESCRIPTION

We discovered that myocardial damage in a subject receiving treatment by extracorporeal membrane oxygenation (ECMO) can be reduced or prevented by administration of a cardioprotective agent to the subject. As explained further below, we discovered that it can be particularly advantageous to administer a cardioprotective agent before ECMO treatment begins. We also discovered that myocardial damage in a subject treated using ECMO can be reduced or prevented by use of normoxemic ECMO, with or without concurrent use of a cardioprotective agent. Additionally, we discovered that myocardial damage in a subject receiving ECMO treatment can be reduced or prevented by preemptive left ventricular (LV) decompression. We further discovered that myocardial damage caused by ECMO can be reduced, prevented, or treated by restoring or maintaining tafazzin levels in heart muscle cells.

Accordingly, the invention provides methods for reducing or preventing myocardial damage in a subject caused by ECMO by administering a cardioprotective agent to the subject and then treating the subject with ECMO, as well as methods for reducing or preventing myocardial damage in a subject caused by ECMO by treating the subject with normoxemic ECMO. The invention also provides methods for reducing or preventing myocardial damage in a subject caused by ECMO by preemptive LV decompression. The invention further provides methods for reducing, preventing, or treating myocardial damage by restoring or maintaining tafazzin levels in heart muscle cells. Additionally, the invention provides systems, composition, and kits for use in carrying out the methods of the invention.

The invention is based, at least in part, on experiments described below in which we discovered that protecting mitochondria in myocardial cells in subjects undergoing ECMO led to reduction or prevention of myocardial damage in such subjects. As a brief background, mitochondria play a central role in myocardial ischemia and reperfusion injury (Boengler et al., Am. J. Physiol. Heart Cir. Physiol. 315(5):H1215-H1231, 2018). Cardiolipin (CL) is a mitochondrial-specific phospholipid and master regulator of mitochondrial integrity in cardiomyocytes, and loss of CL activity accelerates ischemia-reperfusion injury by reducing electron transport chain function, depleting energy production, disrupting mitochondrial integrity, and promoting oxidative stress (Paradies et al., Cells 8(7):728, 2019; Paradies et al., Am. J. Physiol. Heart Circ. Physiol. 315(5):H1341-H1352, 2018). As is explained further in the experimental examples, below, we discovered that VA-ECMO acutely depletes mitochondrial levels of tafazzin, an enzyme that promotes maturation of CL. We also found that loss of both tafazzin and CL activity with VA-ECMO impairs function of multiple electron transport chain complexes, worsens oxidative stress, and increases infarct size compared to reperfusion alone. We additionally discovered that CL activity can be rescued by administration of a CL stabilizing agent, and that delaying coronary reperfusion with VA-ECMO support reduces infarct size, stabilizes electron transport chain function, and reduces oxidative stress as compared to reperfusion alone. We additionally found that use of normoxemic ECMO attenuates oxidative stress and infarct size. Further, we found that preemptive LV decompression decreased pressure-volume area (PVA), decreased infarct size, increased cardioprotective signaling via the reperfusion injury salvage kinase pathway, reduced apoptosis and pro-apoptotic signaling, preserved mitochondrial preserved mitochondrial tafazzin levels, increased mitochondrial cardiolipin levels, and reduced the ratio of immature: mature cardiolipin (MLCL: CL ratio) within the infarct zone as compared to ECMO alone.

The methods, systems, compositions, and kits of the invention are described in further detail below.

### Extracorporeal Membrane Oxygenation (ECMO)

ECMO is an advanced therapy in which blood is temporarily removed from the body for artificial oxygenation of red blood cells and removal of carbon dioxide. The two most common types of ECMO are veno-arterial (VA) ECMO and veno-venous (VV) ECMO. VA ECMO provides support both cardiac and pulmonary support. In VA ECMO blood is drained from the venous system and returned to the arterial system, typically either centrally via a reinjection cannula in the ascending aorta or peripherally via the femoral artery. VV ECMO provides support for isolated pulmonary failure. VV ECMO, blood is drained from the venous system and returned to the venous system using any one of a number of different configurations (e.g., femorofemoral, femorojugular, and single cannulation with double lumen catheters). There are numerous variations possible for both VA- and VV-ECMO approaches that are known in the art. For example, the methods can vary with respect to location of cannula placement, as noted above. In addition, the methods can vary in regard to the level of oxygenation of blood returned to the patient. VA-ECMO, VV-ECMO, and variations of these methods can all be used in the present invention.

ECMO is carried out using an ECMO machine which includes one or more pumps to facilitate movement of the blood via tubing (e.g., polyvinyl chloride tubing), a membrane oxygenator to conduct gas transfer, and a heat exchanger to warm the blood before it is returned to the patient. A patient is connected to an ECMO machine by a physician using standard methods including the placement of cannulae to facilitate removal and return of blood to the body. ECMO machines are available from a number of different commercial sources including, e.g., Medtronic, LivaNova, Maquet, Xenios AG (Fresenius Medical Care), Sorin Group, Terumo, Nipro, and MicroPort, and are available for use in many major hospitals.

### Therapeutic Methods

As explained above, the methods of the invention can be used to reduce or prevent myocardial damage (e.g., left ventricular damage) in subjects undergoing ECMO treatment. In some embodiments, the methods involve administration of one or more cardioprotective agents, which can be done prior to the commencement of ECMO treatment, or by use of normoxemic ECMO, with or without one or more cardioprotective agents. In other embodiments, the methods involve carrying out LV decompression before the commencement of ECMO (i.e., preemptive LV decompression), optionally in the absence of the use of a cardioprotective agent.

Examples of cardioprotective agents that can be used with the methods of the invention are provided below. The cardioprotective agent(s) is administered to subjects using approaches and in amounts that are determined to be appropriate by those of skill in the art. The approaches and amounts may vary based on factors including, for example, the nature of the agent, the condition treated, and the general health of the subject. In some embodiments, the route of administration is intraarterial, intracoronary (e.g., intra-myocaridal, intra-epicardial, or intra-pericardial), intravenous, or oral. In some embodiments, the intraarterial administration is via the ECMO oxygenator circuit and ECMO cannula.

In some embodiments, the cardioprotective agent is administered prior to the commencement of ECMO treatment. For example, the cardioprotective agent can be administered up to 15, 30, 45, or 60 minutes, or before commencement of ECMO treatment. Accordingly, in some embodiments, the cardioprotective agent is administered 5-15, 10-20, 15-25, 20-30, 25-35, 30-40, 35-45, 40-50, 45-55, or 50-60 minutes prior to start of ECMO treatment. In other embodiments, the cardioprotective agent is administered more than one hour prior to commencement of ECMO treatment (e.g., 60-240, 75-180, or 90-120 minutes prior to ECMO start).

In some embodiments, administration of the cardioprotective agent continues during a portion or all of the ECMO treatment. In some embodiments, administration of the cardioprotective agent continues after completion of the ECMO treatment. In some embodiments, administration of the cardioprotective agent is completed before commencement of the ECMO treatment.

In some embodiments, the methods of the invention include carrying out ECMO to generate lower post-oxygenator PaO2 levels (e.g., normoxic PaO2 levels) than is typical in the art. In particular, current VA-ECMO guidelines recommend maintaining a post-oxygenator PaO2 level above 300 mmHg (Extracorporeal Life Support Organizational (ELSO) Guidelines: eslo.org). Based on our studies, such as those described below, the invention provides approaches in which post-oxygenator PaO2 levels are less than 300 mmHg. Accordingly, according to the methods of the invention, in some embodiments the post-oxygenator PaO2 level is less than 300, e.g., less than 275, 250, 225, 200, 175, 150, 125, 120, 100, or 75 mmHg, with the minimum PaO2 level typically being greater than 50 mmHg (e.g., greater than 55, 60, 65, 70, 75, 80, 85, or 90 mmHg). In some embodiments, post-oxygenator PaO2 levels are between 60 and 100 mmHg (e.g., 60-100, 60-90, 60-80, 60-70, 70-100, 70-90, 70-80, 80-100, 80-90, or 90-100 mmHg). In some embodiments, normoxemic ECMO can be considered as a PaO2 of less than 140 mmHg (e.g., less than 120 mmHg). Levels of oxygenation can be set by adjustment of controls on the ECMO machine, as is known by those of skill in the art. Also, PaO2 levels can be detected using methods known in the art. O2 levels can be measured in the ECMO circuit itself (right after the oxygenator) and/or at an arterial site distal from the return cannula (e.g., right radial artery if the cannula is returning oxygenated blood into the femoral artery. Further, these methods, involving the generation of relatively low post-oxygenator PaO2 levels, can be carried out with or without use of a cardioprotective agent as described herein.

The methods of the invention (e.g., the methods described immediately above) can optionally be carried out in conjunction with other interventional treatments including, e.g., coronary artery reperfusion and/or left ventricular decompression.

In some embodiments, the methods described above are carried out in combination with left ventricular (LV) decompression, which typically is commenced before but not after the start of ECMO treatment. In some embodiments, LV decompression is carried out using percutaneous approach such as, for example, an intra-aortic balloon pump (IABP), a trans-valvular pump (TVP), a trans-aortic LV assist device (e.g., Impella®, Abiomed, Danvers, MA; e.g., Impella® 2.5, Impella® CP, Impella® 5.0, Impella® 5.5, Impella® RP, Impella® BTR, or Impella® eCP), trans-aortic drainage catheters, left atrial decompression (e.g., TandemHeart), LV assist, RV assist, Protek Duo, Pulsecath device, Precardia device, ECMO cannulas positioned in the pulmonary artery or thoracic aorta, pulmonary artery drainage cannulas, trans-septal left atrial pulmonary artery and trans-aortic LV venting. In some embodiments, LV decompression is carried out using surgical means, e.g., atrial septostomy or direct surgical LV, LA, and pulmonary artery venting.

In some embodiments, the methods of the invention involve LV decompression or unloading prior to commencement of ECMO treatment, optionally in the absence of the use of a cardioprotective agent. The LV decompression can be carried out using an approach selected to be appropriate by those of skill in the art including, e.g., any of the approaches noted in the immediately preceding paragraph. In some embodiments, the LV decompression is commenced at least 10 minutes, e.g., at least 15, 20, 25, 30, 35, 40, 45, 50, 55, 60, 75, 90, 105, 120, 135, 150, 165, 180, 195, or 210 minutes before ECMO treatment begins. In some embodiments, the LV decompression is carried out no longer than 90, 105, 120, 135, 150, 165, 180, 195, or 210 minutes before ECMO treatment is commenced. Accordingly, the LV decompression can commence, e.g., 10-180, 15-150, 20-120, 25-90, 15-45, 30-60, 45-75, 60-90, 75-105, 90-120, 105-135, 120-150, 135-165, 150-210, 105-195, or 120-180 minutes before commencement of ECMO treatment. In some embodiments, the flow rate of LV decompression is greater than the flow rate of ECMO. For example, in some embodiments, the flow rate for LV decompression is 3-5 L/minute, while the flow rate for ECMO is 2-4 L/minute, provided that the LV decompression flow rate is greater than the ECMO flow rate. For example, the LV decompression flow rate may be 3.5 to 4.5 L/minute, while ECMO flow rate may be 3-4 L/minute, provided that the LV decompression flow rate is greater than the ECMO flow rate. In a specific example, the LV decompression flow rate is 3.5 L/minute, while the ECMO flow rate is 4 L/minute. Once ECMO has begun, combined LV decompression and ECMO treatment is carried out for at least 30, 45, 60, 75, 90, 105, 120, 135, 150, 165, 180, 195, or 210 minutes. In some embodiments, the combined LV decompression and ECMO treatment may be carried out for no more than 90, 105, 120, 135, 150, 165, 180, 195, or 210 minutes. Accordingly, combined LV decompression and ECMO treatment can be carried out, e.g., for 30-60, 45-75, 60-90, 75-105, 90-120, 105-135, 120-150, 135-165, 150-180, 165-195, or 180-210 minutes.

The invention further provides methods of operating medical devices such as those described herein. In one embodiment, a LV decompression device (e.g., as described herein) is operated and an ECMO system (e.g., as described herein) is operated, wherein the LV decompression device is operated prior to commencement of the operation of the ECMO device. The LV decompression device continues to be operated once the operation of the ECMO device commences. The devices are operated in combination in a pumping process. The timing of the operation of the LV decompression device and timing of the operation of the ECMO device can be, e.g., as described above. Furthermore, the relative flow rates of the LV decompression device and the ECMO machine can be, e.g., as described above.

In some embodiments, the methods for reducing or preventing myocardial damage in a subject undergoing ECMO treatment (e.g., the methods described above) are carried out prior to coronary artery reperfusion. In some embodiments, reperfusion begins up to 15, 30, 45, or 60 minutes after commencement of ECMO treatment. Accordingly, in some embodiments, the reperfusion begins 5-15, 10-20, 15-25, 20-30, 25-35, 30-40, 35-45, 40-50, 45-55, or 50-60 minutes after commencement of ECMO treatment. In other embodiments, reperfusion begins more than one hour after commencement of ECMO treatment (e.g., 60-240, 75-180, or 90-120 minutes prior to ECMO start). Any of a number of well-known approaches to reperfusion can be used in conjunction with the methods of the invention. For example, mechanical approaches involving percutaneous coronary intervention (PCI) can be used, which involve the use of balloon angioplasty and stents, as is known in the art. In addition, pharmacologic approaches can be used for reperfusion. Such approaches include, for example, thrombolytic/fibrinolytic therapy, and may optionally utilize agents such as streptokinase (e.g., 1.5 million units over 60 minutes; kabikinase), anistreplase (eminase), alteplase (tPA; recombinant tissue-type plasminogen activator; iv heparin co-administered), reteplase (rPA; recombinant plasminogen activator; nonglycosylated deletion mutant of tPA), tenecteplase (TNK-tPA; multiple point mutant tPA), and urokinase.

In some embodiments, the methods described herein are used to reduce infarct size. In some embodiments, the methods of the invention result in reduction in infarct size by more than 5%, 10%, 20%, 25%, 30%, 40%, 50%, 60%, 75%, 80%, or 90%. Accordingly, in some embodiments, infarct size is reduced by 5-20%, 10-25%, 20-35%, 25-40%, 30-45%, 40-55%, 50-65%, 60-75%, 75-90%, or 80-95%.

The invention also provides methods of reducing, preventing, or treating myocardial damage by restoring or maintaining tafazzin levels in heart muscle cells. This can be achieved by carrying out any one or more of the methods described above. In other embodiments, these methods can involve the use of approaches including, for example, enzyme replacement therapy, gene therapy, mRNA therapy, and CRISPR-based approaches. In enzyme replacement approaches, for example, tafazzin protein can be administered to subjects, while in gene therapy methods DNA, e.g., cDNA, encoding tafazzin, can be administered, optionally by the use of a vector (e.g., an adenoviral vector or an adeno-associated viral vector). In other approaches, direct administration of mRNA (e.g., mRNA encoding tafazzin) can be used. In some embodiments of these methods, the mRNA is modified in order to improve stability and/or reduce immunogenicity. For example, in some embodiments, modified RNA including pseudouridine nucleosides in place of uridine residues is used. In some embodiments, translation is enhanced by the use of a modified cap, e.g., 3′-O-Me-m7G(5′)ppp(5′)G Anti Reverse Cap Analog (ARCA) at the mRNA 5′ end. In other embodiments, proteases that degrade tafazzin can be targeted using, e.g., antisense methods, CRISPR, or small molecules. For example, proteases such as the intermembrane space AAA (i-AAA) and YME1P proteases can be targeted (Claypool et al., J. Cell Biol. 192(3):447-462, 2011). Furthermore, Barth syndrome mutations that cause tafazzin complex lability can be targeted (Claypool et al., supra; Whited et al., Hum. Mol. Gen. 22(3):483-492, 2013). All of these approaches can be carried out before, during, and/or after ECMO treatment, e.g., as described herein, in any combination. Furthermore, these methods and systems can optionally be carried out or used in conjunction with (e.g., prior to) cardiac reperfusion, e.g., as described herein.

### Cardioprotective Agents

Cardioprotective agents for use in the invention include any agent that is known or is found to provide protection to the heart (e.g., the LV) in methods such as those described herein. In some embodiments, the cardioprotective agent reduces infarct size (e.g., as described herein). In some embodiments, the cardioprotective agent is protective of the mitochondria of myocardial cells. In some embodiments, the cardioprotective agent protects cardiolipin (CL) or tafazzin. In some embodiments, the cardioprotective agent targets proteases that degrade tafazzin. In some embodiments, the cardioprotective agent is an antioxidant or an oxygen radical scavenger. Specific, non-limiting examples of cardioprotective agents that can be used in the invention are provided as follows.

In some embodiments, the cardioprotective agent is an aromatic cationic peptide. In some embodiments, the cardioprotective agent is D-Arg-2′,6′-Dmt-Lys-Phe-NH₂ (D-Arginyl-2,6-dimethyl-L-tyrosyl-L-lysyl-L-phenylalaninamide) or a pharmaceutically acceptable salt thereof (e.g., a bis-hydrochloride, tris-hydrochloride, mesylate, tosylate, acetate, or trifluoroacetate salt) (elamipretide, Bendavia, SS-31, TP-131). In some embodiments, the cardioprotective agent is a peptide described in, or falling within a formula of, WO 2014/134562, WO 2017/201433, or WO 2020/131282. Each of these publications is incorporated herein by reference for teachings of such formulae, peptides, and their synthesis. In some embodiments, the cardioprotective agent is a peptide set forth the tables set forth below. In some embodiments, the cardioprotective agent is 2′,6′-dimethyl-Tyr-D-Arg-Phe-Lys-NH₂, Phe-D-Arg-Phe-Lys-NH₂, or 2′,6′-Dmp-D-Arg-Phe-Lys-NH₂, or a pharmaceutically acceptable salt thereof.

In some embodiments, reactive oxygen species (ROS) scavengers can be used. For example, a nitroxide, such as for example Tempol (4-hydroxy-2,2,6,6-tetramethylpiperydine-1-oxyl), or Tiron (4,5-dihydroxy-1,3-benzenedisulfonic acid) can be used.

In some embodiments, mitochondrial protease inhibitors can be used. For example, ClpP inhibitors (e.g., AV167, TG42, TG53, and TG54) and ClpXP inhibitor compounds can be used.

### Subjects and Diseases Treated

Subjects treated in accordance with the invention include any subjects treated with any form of ECMO (e.g., as described above). These subjects include human patients of any age (e.g., adults and elderly subjects, as well as pediatric subjects, including infants). Accordingly, subjects treated with the invention include, e.g., a subject who has or is at risk of developing myocardial infarction, heart failure (e.g., acute heart failure or chronic heart failure), cardiac arrest, heart muscle disease, myocarditis, sepsis, hypothermia, post-transplant complications, cardiogenic shock, cardio-respiratory failure, respiratory failure, lung infection, acute respiratory distress syndrome, pulmonary embolism, congenital diaphragmatic hernia, influenza, meconium aspiration, hantavirus pulmonary syndrome, pulmonary hypertension, pneumonia, respiratory failure, trauma, or Covid-19, or is subject to treatment with or by a ventricular assist device (VAD), high risk percutaneous coronary intervention (PCI), heart transplant, lung transplant, heart surgery, or cardiac catheterization. Furthermore, the invention can be used with subjects on a bridge to recovery (e.g., recovery from surgery including, e.g., heart surgery, such as repair of congenital or degenerative heart defect) or subjects on a bridge to a destination therapy (e.g., transplant, such as heart or lung transplant). Additional subjects that can be treated according to the invention include those being treated for poisoning or poor oxygenation (e.g., due to cystic fibrosis, chronic lung disease, and lung failure).

### Systems, Compositions, and Kits

As noted above, the invention provides systems, compositions, and kits for use in carrying out the methods of the invention. In some embodiments, the systems of the invention include an ECMO machine (e.g., as described herein), together with one or more cardioprotective agent (e.g., as described herein) for use in carrying out the methods of the invention. In some embodiments, a composition or kit of the invention includes one or more disposable element used in or with an ECMO machine (e.g., tubing and/or cannula), together with one or more cardioprotective agent (e.g., as described herein), for use in carrying out the methods of the invention. In some embodiments, the systems of the invention include a left ventricular pump (e.g., as described herein) and an ECMO machine (e.g., as described herein). Optionally, the systems, compositions, or kits include instructions for use of the systems, compositions, or kits for carrying out one or more of the methods described herein.

### EXAMPLES

The following examples are intended to be illustrative only and are not to be considered as limiting the scope of the invention.

### Example 1: Treatment of a Subject Experiencing Myocardial Infarction

A human patient is diagnosed as having experienced a myocardial infarction with evidence of ST elevation in a hospital emergency room. The patient is brought to a catheterization laboratory for primary angioplasty reperfusion. Catheters are delivered via the arm or leg to engage the coronary artery.

In some cases prior to reperfusion, the patient is administered elamipretide (0.05 ug/kg/minute) via intracoronary injection, followed by initiation of VA-ECMO by placement of a cannula in the proximal femoral or jugular vein for drainage of deoxygenated blood, and placement of a cannula in the carotid, femoral, or axillary artery for delivery of newly oxygenated blood to the patient. In other cases, reperfusion may have occurred spontaneously and the drug is delivered followed by VA-ECMO. In other cases, angioplasty may be performed, the drug is delivered, and the angioplasty balloon inflated to recreate occlusion and to allow the drug to penetrate into the myocardium. In each of these cases, the drug is delivered before VA-ECMO initiation.

Some patients may require immediate cannulation for VA-ECMO due to hemodynamic instability. In which case, VA-ECMO is initiated, followed by coronary catheterization and drug delivery either before or immediately after reperfusion.

After reperfusion, VA-ECMO may be continued for a period of 2 hours or more to provide hemodynamic support. The length of time for VA-ECMO treatment is dictated by clinical need and thus can be adjusted by those of skill in the art as needed.

The patient may then require immediate cannulation. In other cases, VA-ECMO may be initiated after delivery of elamipretide and reperfusion therapy. Within 5-30 minutes after elamipretide administration, VA-ECMO treatment is commenced. Within 5-30 minutes after the start of ECMO treatment, coronary angioplasty would be carried out in order to achieve reperfusion of the heart through or around blocked coronary arteries. Alternatively (or in addition to coronary angioplasty), if determined to be appropriate by those of skill in the art, thrombolytic therapy could be used. After completion of reperfusion, ECMO continues for 30 minutes-2 hours, as determined to be appropriate by the attending physician.

### Example 2: Treatment of a Subject Experiencing Myocardial Infarction

A human patient is diagnosed as having experienced a myocardial infarction in a hospital emergency room. The patient is brought to a catheterization laboratory for primary angioplasty reperfusion. Catheters are delivered via the arm or leg to engage the coronary artery. If the patient is hemodynamically unstable and requires increased circulatory support (e.g., increased blood pressure), then VA-ECMO is initiated. For treatment with normoxemic VA-ECMO, a blended gas mixture of oxygen and carbon dioxide is used to achieve an arterial oxygenation level <120 PaO2 mmHg which approximates an arterial oxygen saturation level >95%. Normoxemic levels are maintained while a patient is on VA-ECMO.

### Example 3: Pre-Emptive LV Decompression Limits Infarct Expansion due to VA-ECMO in Models of AMI

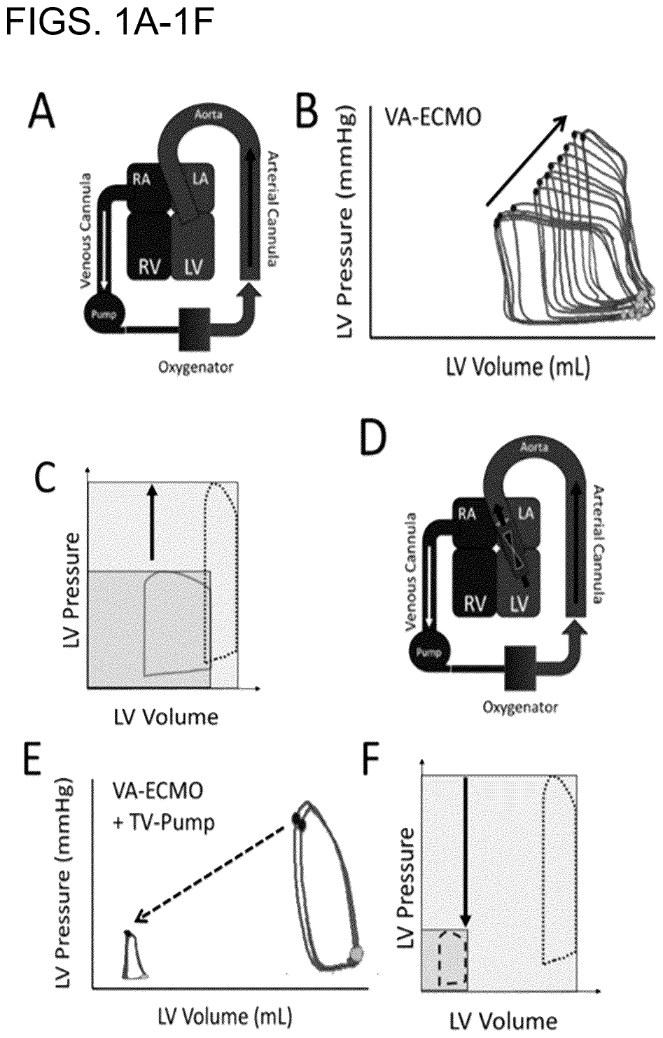

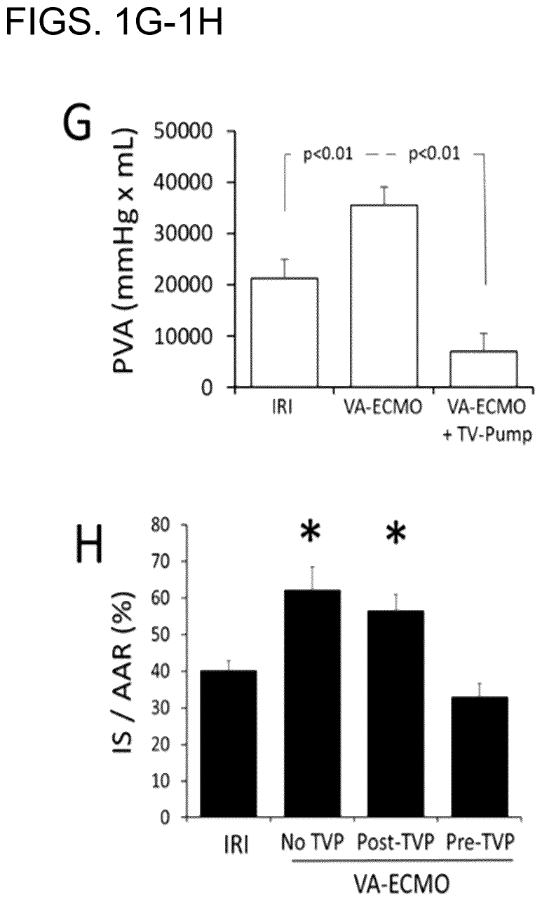

Peripheral VA-ECMO displaces and oxygenates venous blood into the femoral artery thereby driving flow up the aorta, pressurizing the arterial system and increasing LV afterload and wall stress (Bavaria et al., Ann. Thorac. Surg. 45(5):526-532, 1988) (FIGS. 1A-1C). If LV contractility is impaired, as in AMI, higher LV afterload can increase LV pressure and volume (Dickstein, ASAIO J. 64(4):497-501, 2018). Since myocardial oxygen consumption correlates directly with the product of LV pressure and volume, known as the pressure-volume area (PVA), VA-ECMO may increase the LV PVA, thereby worsening oxygen supply-demand mismatch during AMI and increasing myocardial damage (Rao et al., Cir. Heart Fail. 11(9):e004905, 2018). Recent clinical studies suggest that LV distention after initiation of VA-ECMO is associated with poor myocardial recovery, which has led to the use of concomitant pumps to decompress the LV in clinical practice (Patel et al., ASAIO J. 65(1):21-28, 2019; Kapur et al., Cir. Heart Fail. 12(11):e006581, 2019).

We and others established that LV unloading prior to reperfusion is cardioprotective and reduces infarct size in preclinical models of AMI (Kapur et al., JACC Heart Fail. 3(11):873-882, 2015; Esposito et al., J. Am. Coll. Cardiol. 72(5):501-514, 2018; Briceno et al., J. Am. Heart Assoc. 8(22):e013586, 2019; Swain et al., J. Am. Coll. Cardiol. 76(5):684-699, 2020; Kapur et al., Circulation 139(3):337-346, 2019; Meyns et al., J. Am. Coll. Cardiol. 41(7):1087-1095, 2003). We recently reported that compared to reperfusion alone, VA-ECMO or a TVP can reduce LV stroke work, however while the TVP decreased myocardial infarct size, VA-ECMO increased infarct size in a swine model of AMI (Swain et al., J. Am. Coll. Cardiol. 76(5):684-699, 2020). FIGS. 1A-1H illustrate data relating to combined use of TVP and VA-ECMO.

In new data we have now discovered for the first time that pre-emptive TVP decompression for 30 minutes before VA-ECMO initiation preserves levels of tafazzin in the infarct zone. Our data showing that VA-ECMO increases LV PVA and infarct size compared to reperfusion alone in AMI. We also show that VA-ECMO combined with a TVP significantly reduces LV PVA. We show that TVP decompression before, not after VA-ECMO attenuates infarct expansion, but fails to reduce infarct size compared to IRI. If a fraction of the dramatic increase in infarct size with VA-ECMO alone is observed in clinical practice compared to reperfusion alone, 1-year mortality and HF hospitalization are likely to be greatly increased. We have also discovered that pre-emptive LV decompression can be used to protect mitochondrial function and limit VA-ECMO-induced LV injury in AMI with implications for VA-ECMO use in cardiac arrest, advanced HF, and respiratory failure.

A human patient is diagnosed as having experienced a myocardial infarction in a hospital emergency room. The patient is brought to a catheterization laboratory for primary angioplasty reperfusion. Catheters are delivered via the arm or leg to engage the coronary artery. If the patient is hemodynamically unstable and requires increased circulatory support (e.g., increased blood pressure), then VA-ECMO is initiated. In many cases, VA-ECMO may cause an increase in LV pressure or volume overload and subsequent pulmonary congestion. This is especially true for patients with low cardiac function prior to initiation of VA-ECMO. In these cases, a trans-valvular LV decompression pump may be inserted before initiation of VA-ECMO to pre-emptively decompress the LV and activate cardioprotective signaling before initiation of VA-ECMO. LV decompression continues for the duration of VA-ECMO support until no longer needed based on clinical parameters of hemodynamic stability.

### Example 4: Reducing Hyperoxemia during VA-ECMO Attenuates Oxidative Stress and Infarct Size in AMI

Current VA-ECMO treatment guidelines recommend maintaining a post-oxygenator PaO2 above 300 mmHg (Extracorporeal Life Support Organizationa (ELSO) Guidelines: www.elso.org). The more dysfunctional the LV, the more dominant VA-ECMO oxygenation becomes as the mixing point between native and ECMO-driven blood moves towards the aortic root (Chung et al., ScientificWorldJournal 2014:393258). We carried out studies to assess the impact of this hyperoxemic environment during VA-ECMO on coronary blood flow and infarct size.

To interrogate microcirculatory physiology in vivo, pressure and Doppler sensor-tipped guide wires can obtain phasic coronary pressure and flow profiles beyond a coronary occlusion. These data can be used to calculate the collateral flow index (CFI), average peak coronary flow velocity (APV), and the phasic signals post-processed by a technique called wave intensity analysis (WIA), which allows for calculation of the wave energies that accelerate and decelerate coronary blood flow throughout the cardiac cycle (De Silva et al., Circ. Cardiovasc. Interv. 6(2):166-175, 2013; De Silva et al., JACC Cardiovasc. Interv. 7(6):631-640, 2014). Backward expansion wave (BEW), which originates in the intra-myocardial microvasculature during early diastole and accelerates coronary flow, is a dominant factor governing myocardial perfusion (Claridge et al., J. Am. Heart Assoc. 4(12):e002626, 2015). BEW is an index that correlates inversely with myocardial infarct size and is associated with functional myocardial recovery in patients with acute coronary syndromes.

The CFI is calculated by measuring coronary wedge pressure (Pw), right atrial pressure (Pv), and Aortic pressure (Pa) where CFI=(Pw−Pv)/(Pa−Pv). Low CFI values are associated with worsening myocardial ischemia and poor clinical outcomes in AMI (Kim et al., Am. Heart J. 171(1):53-63, 2016; Cuculi et al., J. Am. Coll. Cardiol. 64(18):1894-1904, 2014; Sezer et al., Coron. Artery Dis. 17(2):139-144, 2006). We recently found that LV unloading with a TVP increases the CFI and coronary wedge pressure pulsatility; both of which were associated with reduced infarct size in preclinical models of AMI (Briceno et al., J. Am. Heart Assoc. 8(22):e013586, 2019). We further found that, compared to a TVP, VA-ECMO decreased both the CFI and Pw pulsatility, and increased infarct size. A fundamental effect of VA-ECMO is increased systemic levels of arterial oxygen tension (hyperoxemia). We have studied the impact of ECMO-mediated hyperoxemia on coronary blood flow and infarct size in AMI.

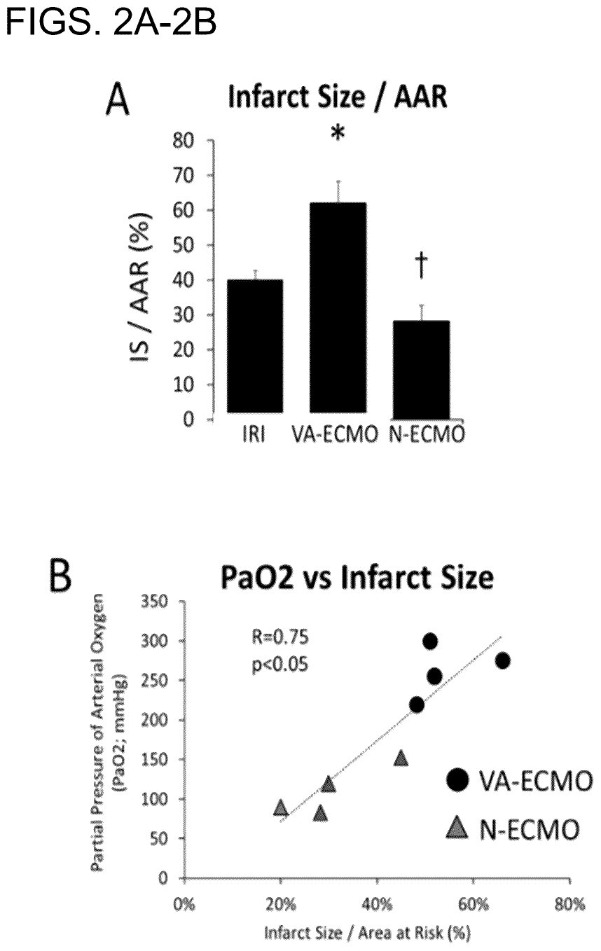

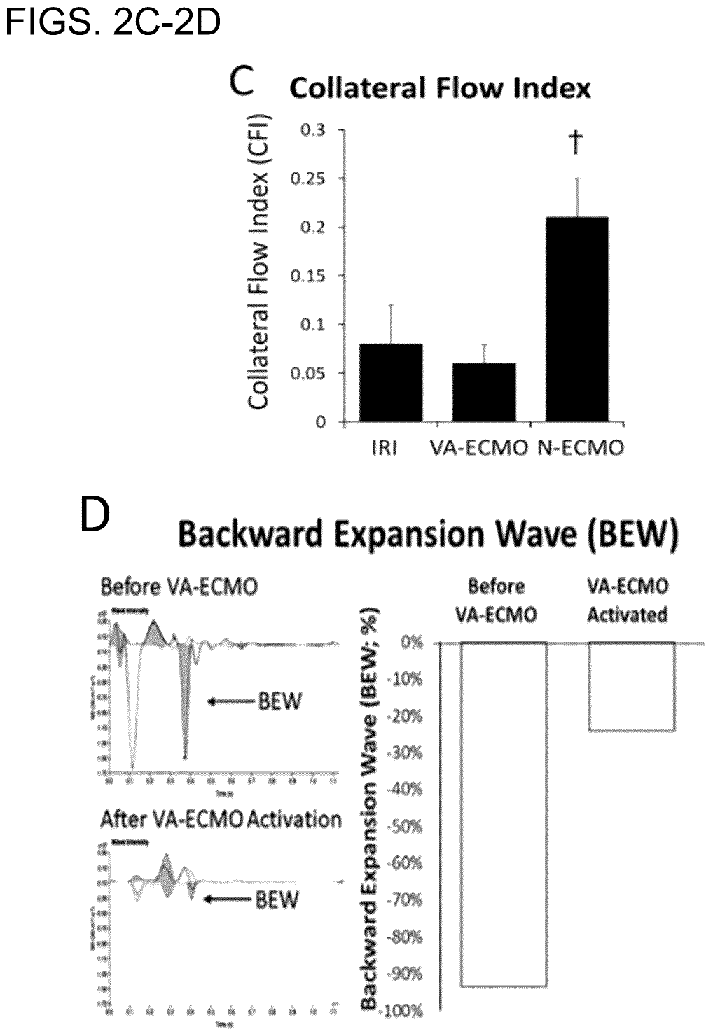

Using a porcine experimental model, we found that targeting a PaO2<120 mmHg with VA-ECMO reduced infarct size by 30% and 55% as compared to IRI or VA-ECMO with a PaO2>200 mmHg, respectively (FIG. 2A). We observed that PaO2 levels during VA-ECMO support are directly associated with infarct size (FIG. 2B). Compared to VA-ECMO with PaO2>200 mmHg, our data show that VA-ECMO with PaO2<120 mmHg increases the CFI and reduces the BEW, showing improved coronary microcirculatory flow (FIG. 2C-D). These findings identify hyperoxemia as a target of therapy to limit infarct expansion due to VA-ECMO. In new and exciting data, we have now discovered that limiting hyperoxemia with VA-ECMO initiation preserves levels of tafazzin (see below) and subsequent mitochondrial function in the infarct zone.

As above, a human patient is diagnosed as having experienced a myocardial infarction in a hospital emergency room. The patient is brought to a catheterization laboratory for primary angioplasty reperfusion. Catheters are delivered via the arm or leg to engage the coronary artery. If the patient is hemodynamically unstable and requires increased circulatory support (e.g., increased blood pressure), then VA-ECMO is initiated. For treatment with normoxemic VA-ECMO, a blended gas mixture of oxygen and carbon dioxide is used to achieve an arterial oxygenation level <120 PaO2 mmHg which approximates an arterial oxygen saturation level >95%. Normoxemic levels are maintained while a patient is on VA-ECMO.

### Example 5: VA-ECMO Depletes Tafazzin and Promotes Mitochondrial Dysfunction in AMI

Mitochondria play a critical role in cardiac metabolism and ischemia-reperfusion injury (Boengler et al., 2018, supra). Cardiolipin (CL) is a master regulator of mitochondrial integrity (Paradies et al., 2019, supra; Paradies, 2018, supra). CL is a diphosphatidylglycerol lipid with a specific affinity for domains of negative membrane curvature and is required for electron transport chain (ETC) function and structural integrity of the inner mitochondrial membrane (IMM). During ischemia-reperfusion injury, total functional CL content can be reduced by decreased biosynthesis, oxidative damage and degradation, and abnormal CL remodeling. CL disruption impairs ETC function and destabilizes ETC super-complex interactions, which reduces energy production, triggers cytochrome-c release from the IMM, promotes apoptosis, and further increases levels of reactive oxygen species (ROS) leading to a vicious cycle of worsening mitochondrial function and myocardial injury (Paradies et al., 2019, supra; Paradies et al., 2018, supra). Oxidized CL can migrate to the outer mitochondrial membrane and interact with microtubule-associated protein 1A/1B-light chain 3 (LC3) to promote autophagy (Dudek et al., Front. Cell Dev. Biol. 61 5:90, 2017).

Tafazzin is a phosphatidyl-lysophospholipid transacylase that is highly expressed in cardiac muscle and catalyzes CL remodeling by converting monolysl-CL (MLCL) to mature CL (Chin et al., J. Dev. Biol. 8(2):10, 2020). Loss of tafazzin activity is associated with an X-linked inherited dilated cardiomyopathy known as Barth Syndrome, which is diagnosed by the presence of a high MLCL:CL ratio (Sabbah, Heart Fail. Rev., 2020). Critical barriers in the field of cardiovascular medicine include the lack of successful cardioprotective strategies to reduce infarct size in AMI beyond reperfusion alone and the lack of mechanistic insight into the impact of VA-ECMO on myocardial ischemia-reperfusion injury.

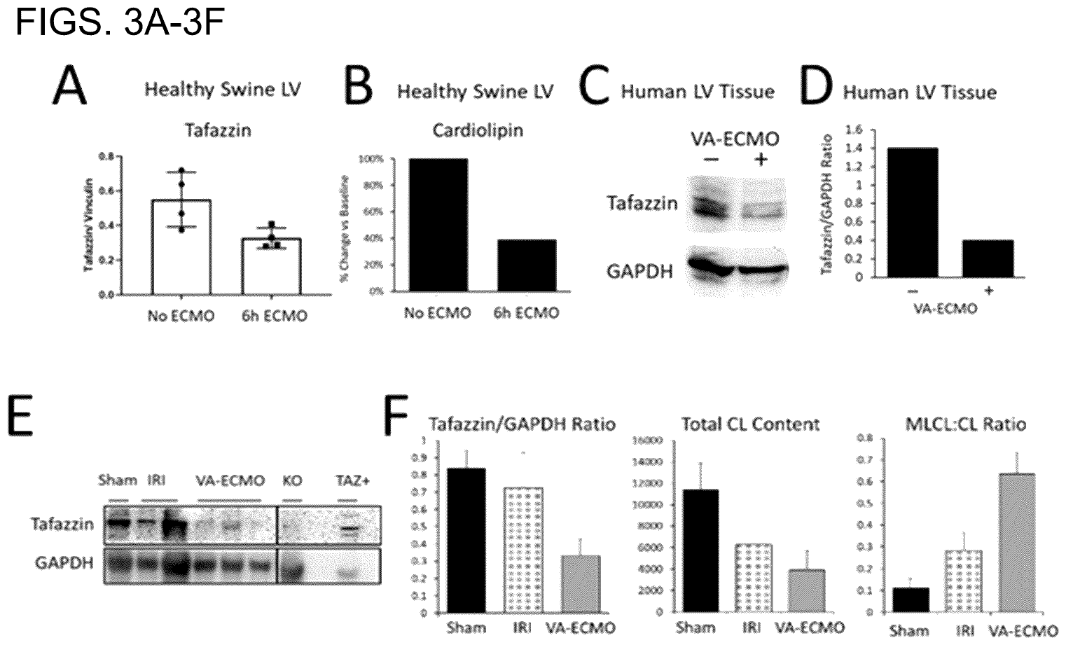

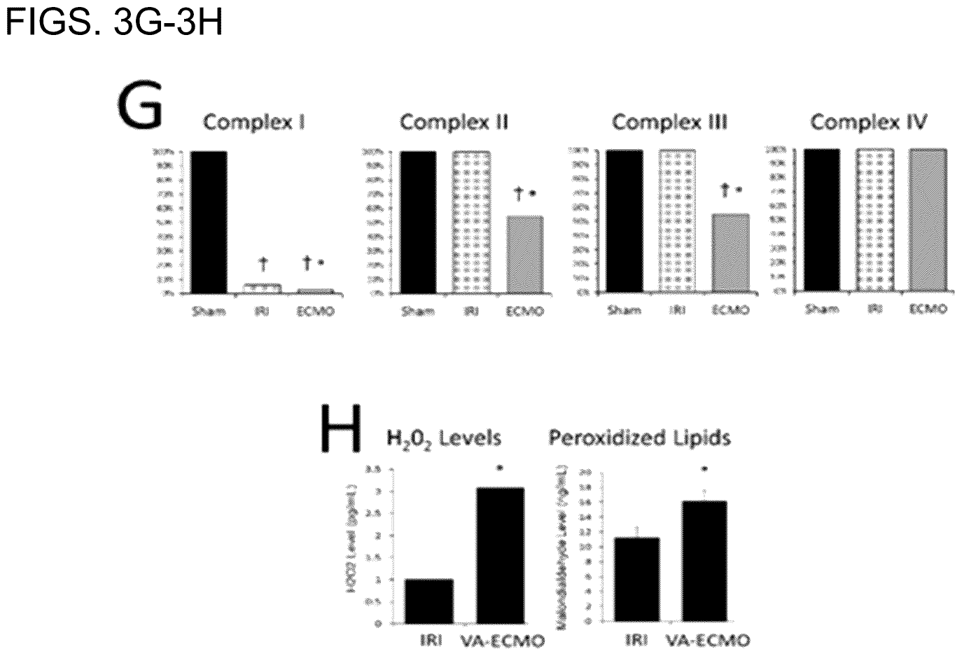

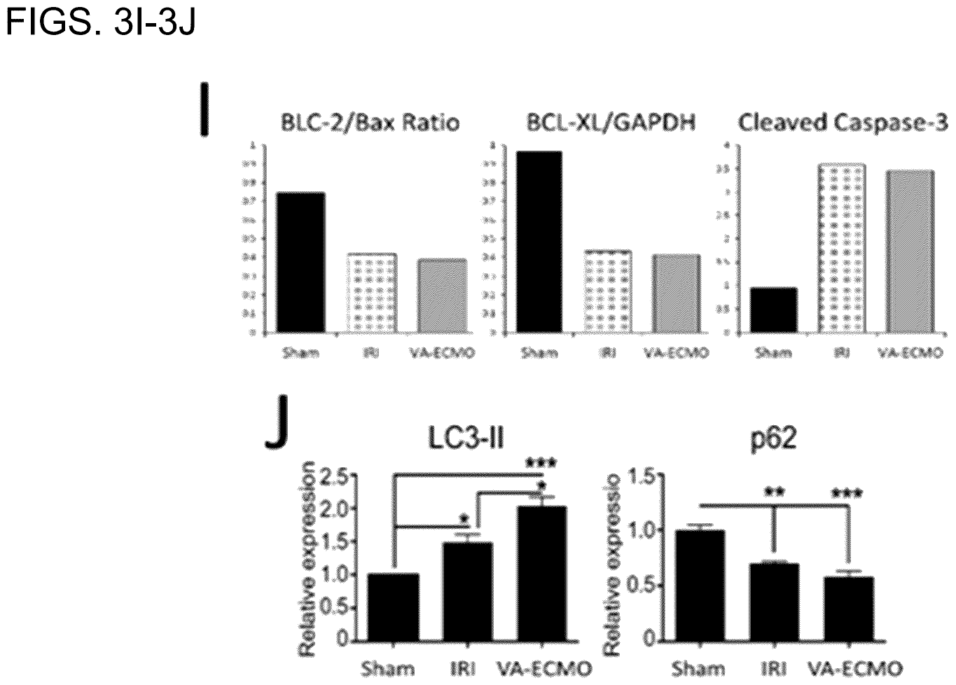

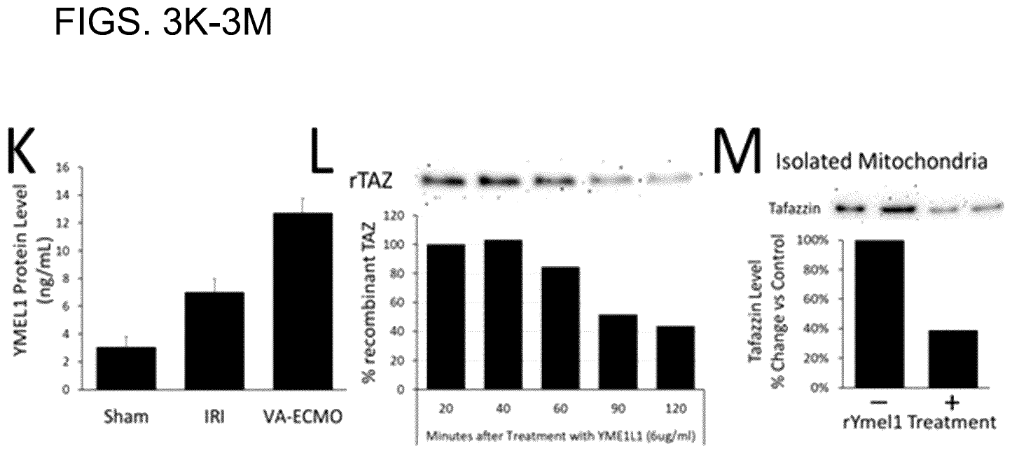

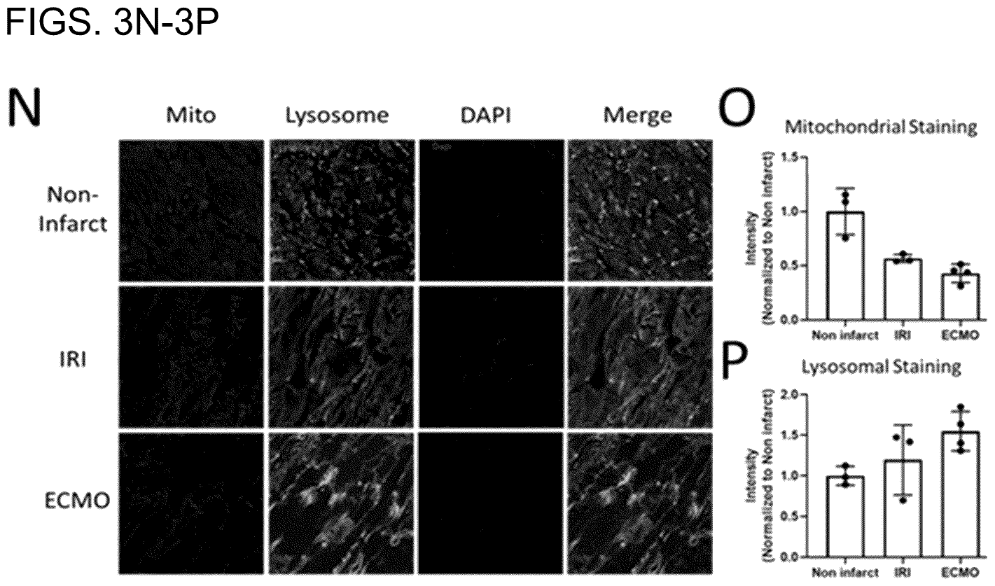

We carried out studies to characterize the impact of VA-ECMO on CL and tafazzin. We found that VA-ECMO directly reduces myocardial tafazzin levels within 6 hours in healthy swine hearts and in human subjects receiving VA-ECMO before cardiac transplantation (FIGS. 3A-3D). We also found that compared to Sham and IRI, VA-ECMO decreases mitochondrial levels of tafazzin and total CL content, and increases the MLCL:CL ratio and decreases function of ETC Complexes I, II, and III (FIGS. 3E-3G). We also found that, compared to IRI, VA-ECMO increases levels of hydrogen peroxide (H202) and peroxidized lipids (malondialdehyde, an end product of lipid peroxidation) within the infarct zone (FIG. 3H). Furthermore, compared to Sham, IRI and VA-ECMO promote a pro-apoptotic environment with reduced BCL-2/BAX ratios and BCL-XL levels and increased cleaved Caspase-3 levels within the infarct zone and further increases levels of LC3 and p62, associated with increased autophagy in the infarct zone (FIGS. 3I and 3J). As described above, we have also identified that pre-emptive LV unloading before VA-ECMO or limiting hyperoxemia with VA-ECMO initiation preserves levels of tafazzin in the infarct zone. These findings have identified novel mechanisms by which VA-ECMO destabilizes mitochondrial integrity during IRI and show that targeting CL may limit LV damage during VA-ECMO support in AMI.

We also have found that VA-ECMO increases levels of the mitochondrial ATPase associated (iAAA) protease Ymel1 in LV lysates from the infarct zone (FIG. 3K) and have shown for the first time that Ymel1 rapidly degrades tafazzin in a time-dependent manner in vitro and in isolated mitochondria (FIGS. 3K-3M). These findings suggest a new mechanism for VA-ECMO mediated mitochondrial damage and identify Ymel1 as a novel potential target of therapy. In additional studies, confocal microscopy with mitochondrial specific markers and lysosomal staining showed that VA-ECMO decreases mitochondrial content, increases CM lysosomal expression by 27%, and increases localization with mitochondria and cardiomyocyte nuclei (FIGS. 3N-3P).

### Example 6: Targeting Cardiolipin during VA-ECMO support May Reduce Infarct Size in AMI

Elamipretide is an amphipathic tetrapeptide that interacts with CL to stabilize the IMM, enhance ETC function, and limit mitochondrial ROS production. A clinical trial of elamipretide in humans (EMBRACE-STEMI Trial) showed that IV elamipretide given before reperfusion for anterior ST-segment elevation myocardial infarction failed to reduce infarct size (Gibson et al., Eur. Heart J. 37(16):1296-1303, 2016), in contrast to prior findings in animal models (Kloner et al., J. Am. Heart Assoc. 1(3):e001644, 2012). We carried out studies to explore whether the combination of intracoronary (IC) delivery of elamipretide and delayed reperfusion with VA-ECMO circulatory support may improve mitochondrial function, limit reperfusion injury, and reduce both infarct expansion due to VA-ECMO and total infarct size compared to reperfusion alone in AMI.

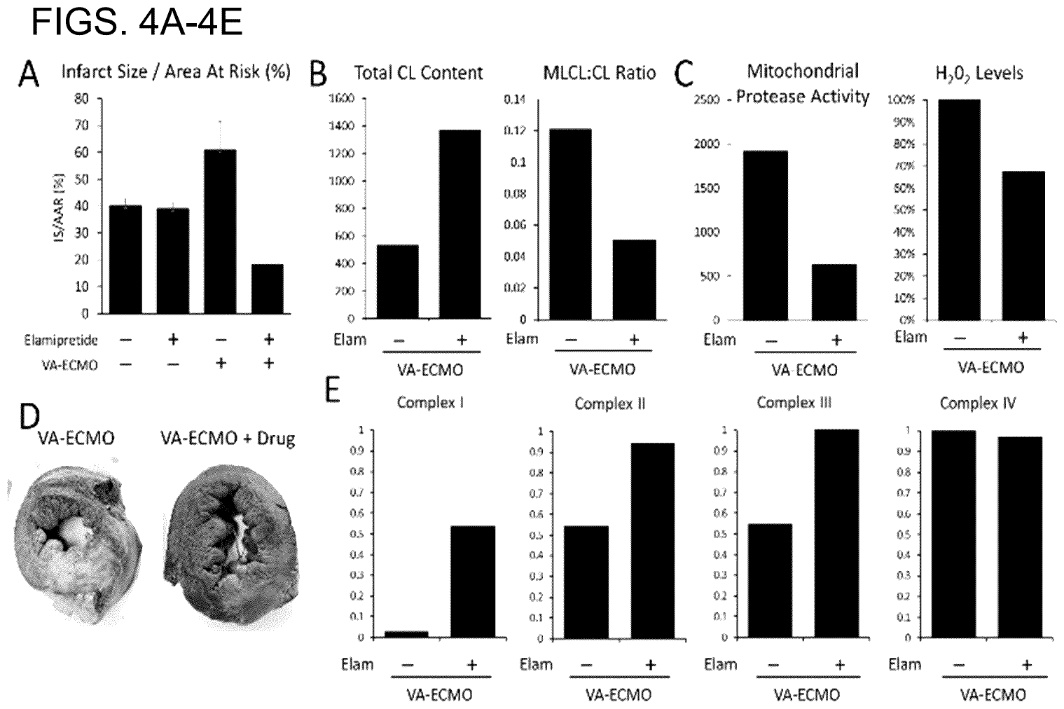

To begin exploring whether stabilizing CL activity can limit myocardial infarct size during VA-ECMO support for AMI, we employed an over-the-wire coronary angioplasty balloon to delivery elamipretide into the area at risk while maintaining occlusion of the LAD. We treated adult male swine with a single IC injection of either elamipretide (0.05 ug/kg/minute IC over 10 minutes) or vehicle prior to initiating VA-ECMO and then delayed coronary reperfusion for 30 minutes, thereby allowing the drug to reach the area at risk and stabilize CL. Elamipretide was continued as an IV infusion (0.05 ug/kg/minute) during 180 minutes of reperfusion. Compared to vehicle controls, elamipretide reduced infarct size by 70% compared to IRI plus VA-ECMO support and by 55% and decreased the MLCL:CL ratio, mitochondrial protease activity, and H202 levels in the infarct zone (FIGS. 4B and 4C) and further improved ETC complex function (FIG. 4E) as compared to IRI alone (FIGS. 4A and 4D). Elamipretide treatment without VA-ECMO failed to reduce infarct size compared to IRI alone (FIG. 4A). Elamipretide dosing was based on prior reports (Kloner et al., J. Am. Heart Assoc. 1(3):e001644, 2012; Gibson et al., Eur. Heart J. 37(16):1296-1303, 2016). These data identify a new therapeutic approach whereby IC elamipretide and delaying reperfusion for 30 minutes while systemic blood pressure is supported with VA-ECMO stabilizes CL content, reduces infarct size, and preserves mitochondrial ETC function.

### Example 7: Cardioprotective Agent Administration Prior to ECMO Treatment Improves Survival and Functional Outcomes after AMI

Despite timely reperfusion, recent studies identified that approximately 15-40% of the LV is damaged after AMI (Stone et al., J. Am Coll. Cardiol. 67(14):1674-1683, 2016). Reducing infarct size is a well-established target of therapy to improve short and long-term clinical outcomes including mortality and HF hospitalization. Current cardioprotective strategies under investigation include ischemic conditioning, systemic hypothermia, and pharmacologic approaches to limit reperfusion injury. Although promising, one of the most critical barriers to successful implementation of these strategies is the mandate for rapid coronary reperfusion to limit ischemic injury and therefore insufficient time for any cardioprotective therapy to affect myocardial injury zones. There exists a need for improved strategies to protect mitochondrial function while stabilizing systemic hemodynamics before reperfusion.

We recently reported that LV unloading with a TVP and delaying reperfusion for 30-60 minutes reduces infarct size in preclinical models of AMI (Esposito et al., J. Am. Coll. Cardiol. 72(5):501-514, 2018; Swain et al., J. Am. Coll. Cardiol. 76(5):684-699, 2020). We tested this approach in a pilot clinical trial and observed that LV unloading and delayed reperfusion may reduce infarct size compared to LV unloading and immediate reperfusion, which is now being tested in a large pivotal clinical trial comparing LV unloading and delayed reperfusion to reperfusion alone (Kapur et al., Circulation 139(3):337-346, 2019; Clinical Trials.Gov Identifier: NCT03947619). Our preliminary data suggest that VA-ECMO increases LV wall stress and causes infarct expansion. However, VA-ECMO is widely used around the world due to low cost, ease of implantation, and versatility as a circulatory and respiratory support pump. VA-ECMO use is growing and current trials are testing its utility in AMI. Based on the data described above, we identified that VA-ECMO may increase infarct size by impairing CL activity—a master regulator of mitochondrial structure and function. We further show that treatment with elamipretide before initiation of VA-ECMO stabilizes CL and significantly reduces infarct size compared to reperfusion without VA-ECMO. Elamipretide treatment without VA-ECMO did not reduce infarct size, suggesting that IC delivery and delaying reperfusion while on VA-ECMO support may be necessary and sufficient to optimize cardioprotection. Accordingly, our data support the combination of VA-ECMO and elamipretide as a cardioprotective approach to limit reperfusion injury by protecting mitochondrial function in AMI.

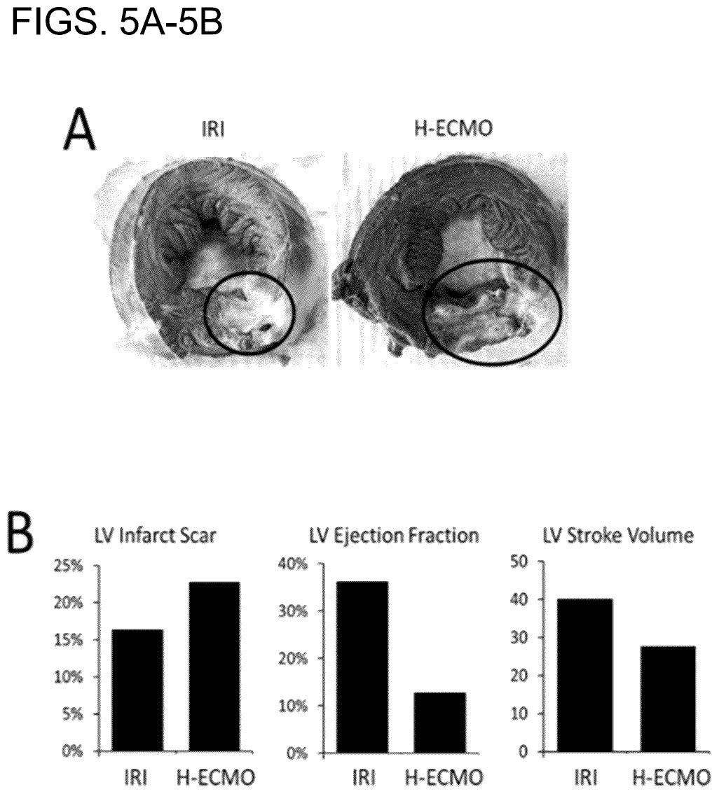

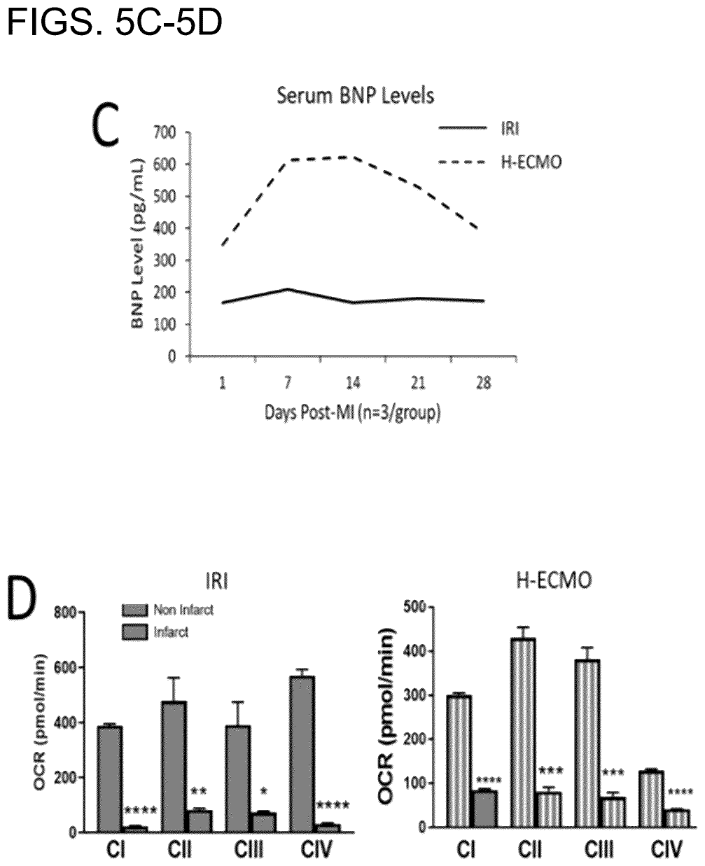

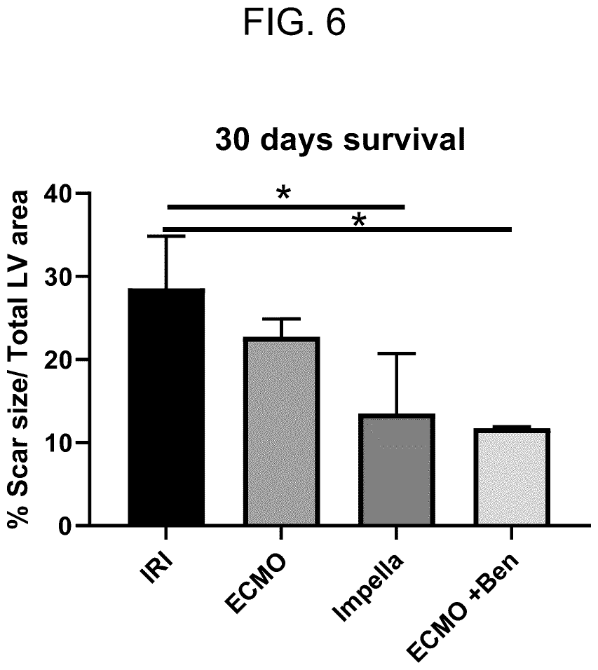

To explore late-term outcomes after VA-ECMO with or without cardioprotective drug therapy in AMI, we decannulated, recovered, and monitored swine for 28 days after peripheral cannulation for VA-ECMO support. Adult male swine were subjected to coronary occlusion followed by reperfusion alone or VA-ECMO before reperfusion for 180 minutes. In a separate group, swine were treated with a cardioprotective agent before initiation of VA-ECMO with a 30-minute delay to reperfusion. After 28 days, we identified for the first time that compared to reperfusion alone, VA-ECMO increases infarct scar size and impairs LV function after AMI (FIGS. 5A and 5B). We further identified that compared to reperfusion, VA-ECMO treatment increased circulating brain-natriuretic peptide (BNP) levels and failed to recover ETC complex function weeks after MI, suggesting worse HF (FIGS. 5C and 5D). These findings support our central hypothesis that VA-ECMO promotes LV scar after AMI and further that treatment with a cardioprotective agent reduces LV scar size 30 days after AMI, which may have broad reaching implications for any patient with acute myocardial infarction or patients at risk of developing cardiac impairment while receiving VA-ECMO. Broad groups of patients may be treated using this approach including, e.g., all AMI patients and all patients treated with VA-ECMO. Indications include, for example, cardiogenic shock, heart failure, lung failure, myocarditis, trauma, and arrhythmia. In additional studies, we have shown that the combination of a cardioprotective stabilizing agent (i.e., elamipretide aka Bendavia, SS-31, or TP-131; D-Arg-dimethylTyr-Lys-Phe-NH₂) given before VA-ECMO can limit LV scar size and late-term LV injury. Compared to reperfusion alone or VA-ECMO alone before reperfusion, VA-ECMO+cardioprotective drug (i.e., elamipretide aka Bendavia) treatment reduced infarct scar size at 30 days. (FIG. 6).

### Example 8: Cardioprotection by Preemptive LV Decompression Followed by ECMO

Addition of Impella CP (LV decompression pump) to veno-arterial extracorporeal membrane oxygenation (ECMO) is referred to herein as EC-Pella. We have determined that compared to ECMO alone, EC-Pella mitigates left ventricular (LV) loading when Impella is activated prior to ECMO before reperfusion in AMI, known as pre-emptive ECPella.

Ischemia-reperfusion injury (IRI) was induced in adult swine via percutaneous occlusion of the left anterior descending artery (LAD) for 90 minutes, followed by activation of either ECMO alone for 30 minutes or EC-Pella for 45 minutes with persistent LAD occlusion. All groups underwent 180 minutes of reperfusion (n=4-6/group). To study whether the sequence of device activation impacts infarct size, we compared activation of ECMO alone for 30 minutes followed by simultaneous Impella CP for 15 minutes (Bailout EC-Pella) versus Impella CP activation for 30 minutes followed by simultaneous ECMO for 15 minutes (Pre-emptive EC-Pella) before reperfusion. LV hemodynamics and ventriculo-arterial (VA) coupling were assessed.

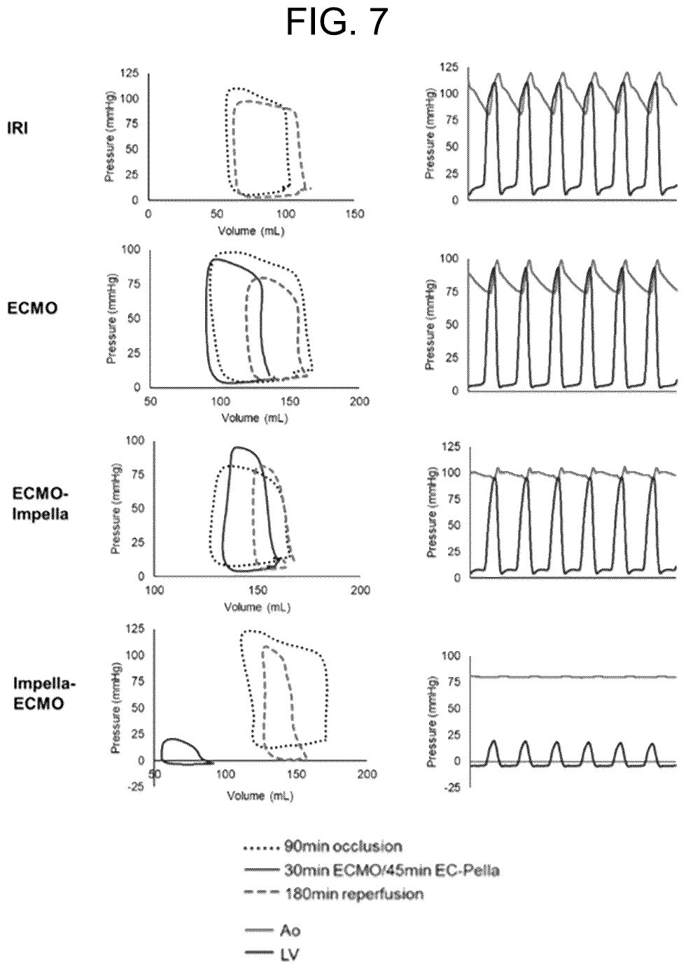

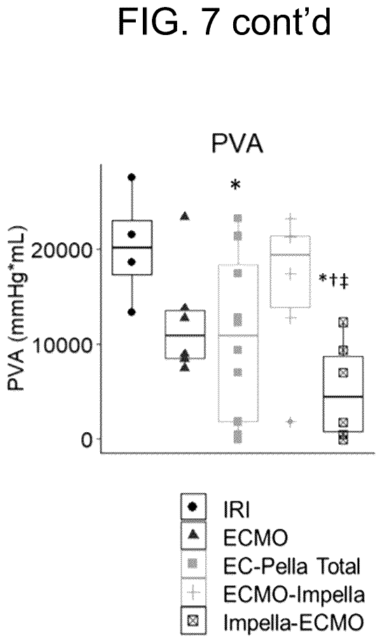

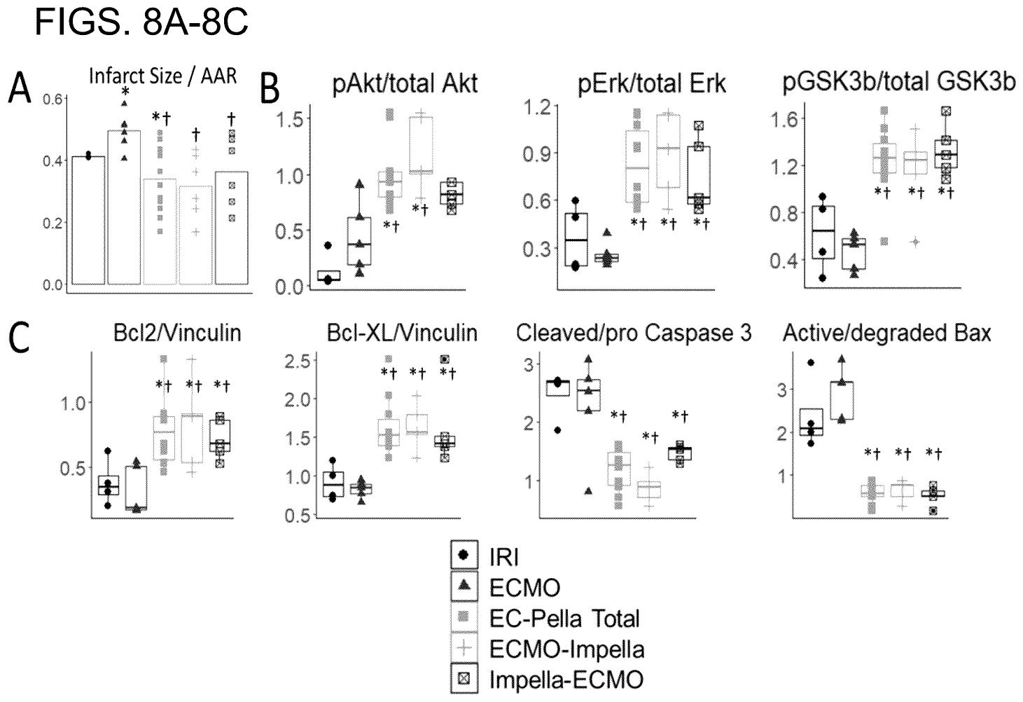

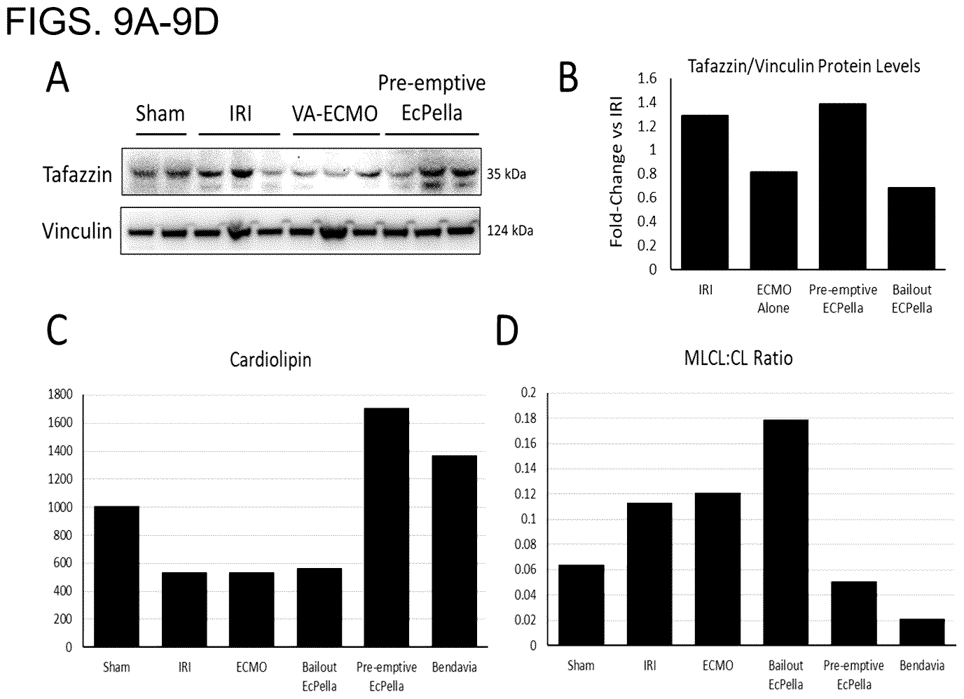

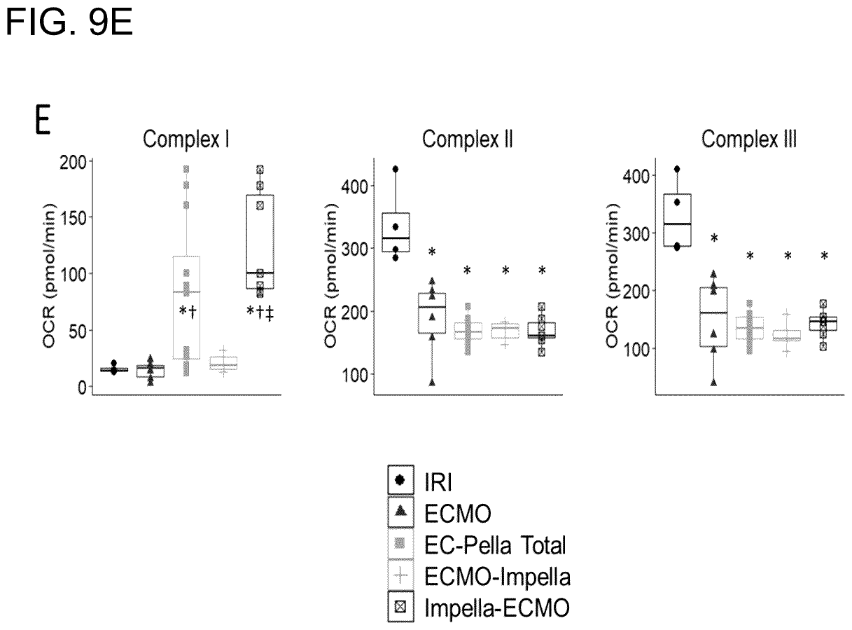

Compared to IRI and ECMO alone, EC-Pella decreased PVA when applied as pre-emptive ECPella but not Bailout ECPella (FIG. 7). Both EC-Pella configurations decreased infarct size (FIG. 8A), increased cardioprotective signaling via the reperfusion injury salvage kinase pathway (FIG. 8B), and reduced apoptosis and pro-apoptotic signaling (FIG. 8C). Pre-emptive, but not bailout, ECPella preserved mitochondrial preserved mitochondrial tafazzin levels (FIGS. 9A-B), increased mitochondrial cardiolipin levels (FIG. 9C), and reduced the ratio of immature: mature cardiolipin (MLCL: CL ratio; FIG. 9D) within the infarct zone compared to ECMO alone. Treatment with elamipretide (Bendavia) increases cardiolipin and reduces mono-lysl cardiolipin: mature cardiolipin (MLCL: CL) ratios to a similar degree as pre-emptive ECPella (FIGS. 9C-9D). Pre-emptive ECPella also increased mitochondrial Complex I activity compared to ECMO or reperfusion alone, but Bailout ECPella did not (FIG. 9E).

We report for the first time that compared to ECMO alone, combining ECMO and Impella (ECPella) in any sequence reduces infarct size, increases cardioprotective signaling and decreases apoptosis. Initiation of EC-Pella with Impella prior to ECMO (pre-emptive ECPella) preserves mitochondrial tafazzin and mature cardiolipin levels as well as preserves Complex 1 function, but bailout ECPella does not. As used in this example, ECMO-Impella=Bailout ECPella=Impella is applied after ECMO initiation, and Impella-ECMO=Pre-emptive ECPella=Impella is applied before ECMO initiation.

TABLE 1
Exemplary Peptides

| | |
|---|---|
| 1 | 2′,6′-Dmp-D-Arg-2′,6′-Dmt-Lys-NH₂ |
| 2 | 2′,6′-Dmp-D-Arg-Phe-Lys-NH₂ |
| 3 | 2′,6′-Dmt-D-Arg-PheOrn-NH₂ |
| 4 | 2′,6′-Dmt-D-Arg-Phe-Ahp(2-aminoheptanoicacid)-NH₂ |
| 5 | 2′,6′-Dmt-D-Arg-Phe-Lys-NH₂ |
| 6 | 2′,6′-Dmt-D-Cit-PheLys-NH₂ |
| 7 | Ala-D-Phe-D-Arg-Tyr-Lys-D-Trp-His-D-Tyr-Gly-Phe |
| 8 | Arg-D-Leu-D-Tyr-Phe-Lys-Glu-D-Lys-Arg-D-Trp-Lys-D-Phe-Tyr-D-Arg-Gly |
| 9 | Asp-Arg-D-Phe-Cys-Phe-D-Arg-D-Lys-Tyr-Arg-D-Tyr-Trp-D-His-Tyr-D-Phe-Lys-Phe |
| 10 | Asp-D-Trp-Lys-Tyr-D-His-Phe-Arg-D-Gly-Lys-NH₂ |
| 11 | D-Arg-2′,6′-Dmt-Lys-Phe-NH₂ |
| 12 | D-Glu-Asp-Lys-D-Arg-D-His-Phe-Phe-D-Val-Tyr-Arg-Tyr-D-Tyr-Arg-His-Phe-NH₂ |
| 13 | D-His-Glu-Lys-Tyr-D-Phe-Arg |
| 14 | D-His-Lys-Tyr-D-Phe-Glu-D-Asp-D-Asp-D-His-D-Lys-Arg-Trp-NH₂ |
| 15 | D-Tyr-Trp-Lys-NH₂ |
| 16 | Glu-Arg-D-Lys-Tyr-D-Val-Phe-D-His-Trp-Arg-D-Gly-Tyr-Arg-D-Met-NH₂ |
| 17 | Gly-Ala-Lys-Phe-D-Lys-Glu-Arg-Tyr-His-D-Arg-D-Arg-Asp-Tyr-Trp-D-His-Trp-His-D-Lys-Asp |
| 18 | Gly-D-Phe-Lys-His-D-Arg-Tyr-NH₂ |
| 19 | His-Tyr-D-Arg-Trp-Lys-Phe-D-Asp-Ala-Arg-Cys-D-Tyr-His-Phe-D-Lys-Tyr-His-Ser-NH₂ |
| 20 | Lys-D-Arg-Tyr-NH₂ |
| 21 | Lys-D-Gln-Tyr-Arg-D-Phe-Trp-NH₂ |
| 22 | Lys-Trp-D-Tyr-Arg-Asn-Phe-Tyr-D-His-NH₂ |
| 23 | Met-Tyr-D-Arg-Phe-Arg-NH₂ |
| 24 | Met-Tyr-D-Lys-Phe-Arg |
| 25 | Phe-Arg-D-His-Asp |
| 26 | Phe-D-Arg-2′,6′-Dmt-Lys-NH₂ |
| 27 | Phe-D-Arg-His |
| 28 | Phe-D-Arg-Lys-Trp-Tyr-D-Arg-His |
| 29 | Phe-D-Arg-Phe-Lys-NH₂ |
| 30 | Phe-Phe-D-Tyr-Arg-Glu-Asp-D-Lys-Arg-D-Arg-His-Phe-NH₂ |
| 31 | Phe-Tyr-Lys-D-Arg-Trp-His-D-Lys-D-Lys-Glu-Arg-D-Tyr-Thr |
| 32 | Thr-Gly-Tyr-Arg-D-His-Phe-Trp-D-His-Lys |
| 33 | Thr-Tyr-Arg-D-Lys-Trp-Tyr-Glu-Asp-D-Lys-D-Arg-His-Phe-D-Tyr-Gly-Val-Ile-D-His-Arg- |
| | Tyr-Lys-NH₂ |
| 34 | Trp-D-Lys-Tyr-Arg-NH₂ |
| 35 | Trp-Lys-Phe-D-Asp-Arg-Tyr-D-His-Lys |
| 36 | Tyr-Asp-D-Lys-Tyr-Phe-D-Lys-D-Arg-Phe-Pro-D-Tyr-His-Lys |
| 37 | Tyr-D-Arg-Phe-Lys-Glu-NH₂ |
| 38 | Tyr-D-Arg-Phe-Lys-NH₂ |
| 39 | Tyr-D-His-Phe-D-Arg-Asp-Lys-D-Arg-His-Trp-D-His-Phe |
| 40 | Tyr-His-D-Gly-Met |
| 41 | Val-D-Lys-His-Tyr-D-Phe-Ser-Tyr-Arg-NH₂ |

TABLE 2

| Amino | Amino | Amino | Amino | |
|---|---|---|---|---|
| Acid | Acid | Acid | Acid | C-Terminal |
| Position 1 | Position 2 | Position 3 | Position 4 | Modification |
| Tyr | D-Arg | Phe | Lys | NH₂ |
| Tyr | D-Arg | Phe | Orn | NH₂ |
| Tyr | D-Arg | Phe | Dab | NH₂ |
| Tyr | D-Arg | Phe | Dap | NH₂ |
| 2′6′Dmt | D-Arg | Phe | Lys | NH₂ |
| 2′6′Dmt | D-Arg | Phe | Lys-NH(CH₂)₂- | NH₂ |
| | | | NH-dns | |
| 2′6′Dmt | D-Arg | Phe | Lys-NH(CH₂)₂- | NH₂ |
| | | | NH-atn | |
| 2′6′Dmt | D-Arg | Phe | dnsLys | NH₂ |
| 2′6′Dmt | D-Cit | Phe | Lys | NH₂ |
| 2′6′Dmt | D-Cit | Phe | Ahp | NH₂ |
| 2′6′Dmt | D-Arg | Phe | Orn | NH₂ |
| 2′6′Dmt | D-Arg | Phe | Dab | NH₂ |
| 2′6′Dmt | D-Arg | Phe | Dap | NH₂ |
| 2′6′Dmt | D-Arg | Phe | Ahp(2-amino | NH₂ |
| | | | heptanoic acid) | |
| Bio-2′6′Dmt | D-Arg | Phe | Lys | NH₂ |
| 3′5′Dmt | D-Arg | Phe | Lys | NH₂ |
| 3′5′Dmt | D-Arg | Phe | Orn | NH₂ |
| 3′5′Dmt | D-Arg | Phe | Dab | NH₂ |
| 3′5′Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′6′Dmt | D-Arg | Tyr | Lys | NH₂ |
| 2′6′Dmt | D-Arg | Tyr | Orn | NH₂ |
| 2′6′Dmt | D-Arg | Tyr | Dab | NH₂ |
| 2′6′Dmt | D-Arg | Tyr | Dap | NH₂ |
| 2′6′Dmt | D-Arg | 2′6′Dmt | Lys | NH₂ |
| 2′6′Dmt | D-Arg | 2′6′Dmt | Orn | NH₂ |
| 2′6′Dmt | D-Arg | 2′6′Dmt | Dab | NH₂ |
| 2′6′Dmt | D-Arg | 2′6′Dmt | Dap | NH₂ |
| 3′5′Dmt | D-Arg | 3′5′Dmt | Arg | NH₂ |
| 3′5′Dmt | D-Arg | 3′5′Dmt | Lys | NH₂ |
| 3′5′Dmt | D-Arg | 3′5′Dmt | Orn | NH₂ |
| 3′5′Dmt | D-Arg | 3′5′Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′6′Dmt | D-Lys | Phe | Dab | NH₂ |
| 2′6′Dmt | D-Lys | Phe | Dap | NH₂ |
| 2′6′Dmt | D-Lys | Phe | Arg | NH₂ |
| 2′6′Dmt | D-Lys | Phe | Lys | NH₂ |
| 3′5′Dmt | D-Lys | Phe | Orn | NH₂ |
| 3′5′Dmt | D-Lys | Phe | Dab | NH₂ |
| 3′5′Dmt | D-Lys | Phe | Dap | NH₂ |
| 3′5′Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′6′Dmt | D-Lys | Tyr | Lys | NH₂ |
| 2′6′Dmt | D-Lys | Tyr | Orn | NH₂ |
| 2′6′Dmt | D-Lys | Tyr | Dab | NH₂ |
| 2′6′Dmt | D-Lys | Tyr | Dap | NH₂ |
| 2′6′Dmt | D-Lys | 2′6′Dmt | Lys | NH₂ |
| 2′6′Dmt | D-Lys | 2′6′Dmt | Orn | NH₂ |
| 2′6′Dmt | D-Lys | 2′6′Dmt | Dab | NH₂ |
| 2′6′Dmt | D-Lys | 2′6′Dmt | Dap | NH₂ |
| 2′6′Dmt | D-Arg | Phe | dnsDap | NH₂ |
| 2′6′Dmt | D-Arg | Phe | atnDap | NH₂ |
| 3′5′Dmt | D-Lys | 3′5′Dmt | Lys | NH₂ |
| 3′5′Dmt | D-Lys | 3′5′Dmt | Orn | NH₂ |
| 3′5′Dmt | D-Lys | 3′5′Dmt | Dab | NH₂ |
| 3′5′Dmt | D-Lys | 3′5′Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′6′Dmt | D-Arg | Phe | Arg | NH₂ |
| 2′6′Dmt | D-Lys | Phe | Arg | NH₂ |
| 2′6′Dmt | D-Orn | Phe | Arg | NH₂ |
| 2′6′Dmt | D-Dab | Phe | Arg | NH₂ |
| 3′5′Dmt | D-Dap | Phe | Arg | NH₂ |
| 3′5′Dmt | D-Arg | Phe | Arg | NH₂ |
| 3′5′Dmt | D-Lys | Phe | Arg | NH₂ |
| 3′5′Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′6′Dmt | D-Arg | 2′6′Dmt | Arg | NH₂ |
| 2′6′Dmt | D-Lys | 2′6′Dmt | Arg | NH₂ |
| 2′6′Dmt | D-Orn | 2′6′Dmt | Arg | NH₂ |
| 2′6′Dmt | D-Dab | 2′6′Dmt | Arg | NH₂ |
| 3′5′Dmt | D-Dap | 3′5′Dmt | Arg | NH₂ |
| 3′5′Dmt | D-Arg | 3′5′Dmt | Arg | NH₂ |
| 3′5′Dmt | D-Lys | 3′5′Dmt | Arg | NH₂ |
| 3′5′Dmt | D-Orn | 3′5′Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |

Dab = diaminobutyric
Dap = diaminopropionic acid
Dmt = dimethyltyrosine
Mmt = 2′-methyltyrosine
Tmt = N,2′,6′-trimethyltyrosine
Hmt = 2′-hydroxy,6′-methyltyrosine
dnsDap = β-dansyl-L-α,β-diaminopropionic acid
atnDap = β-anthraniloyl-L-α,β-diaminopropionic acid
Bio = biotin

TABLE 3

| Amino Acid | Amino Acid | Amino Acid | Amino Acid | C-Terminal |
|---|---|---|---|---|
| Position 1 | Position 2 | Position 3 | Position 4 | Modification |
| D-Arg | Dmt | Lys | Phe | NH₂ |
| D-Arg | Dmt | Phe | Lys | NH₂ |
| D-Arg | Phe | Lys | Dmt | NH₂ |
| D-Arg | Phe | Dmt | Lys | NH₂ |
| D-Arg | Lys | Dmt | Phe | NH₂ |
| D-Arg | Lys | Phe | Dmt | NH₂ |
| Phe | Lys | Dmt | D-Arg | NH₂ |
| Phe | Lys | D-Arg | Dmt | NH₂ |
| Phe | D-Arg | Phe | Lys | NH₂ |
| Phe | D-Arg | Dmt | Lys | NH₂ |
| Phe | D-Arg | Lys | Dmt | NH₂ |
| Phe | Dmt | D-Arg | Lys | NH₂ |
| Phe | Dmt | Lys | D-Arg | NH₂ |
| Lys | Phe | D-Arg | Dmt | NH₂ |
| Lys | Phe | Dmt | D-Arg | NH₂ |
| Lys | Dmt | D-Arg | Phe | NH₂ |
| Lys | Dmt | Phe | D-Arg | NH₂ |
| Lys | D-Arg | Phe | Dmt | NH₂ |
| Lys | D-Arg | Dmt | Phe | NH₂ |
| D-Arg | Dmt | D-Arg | Phe | NH₂ |
| D-Arg | Dmt | D-Arg | Dmt | NH₂ |
| D-Arg | Dmt | D-Arg | Tyr | NH₂ |
| D-Arg | Dmt | D-Arg | Trp | NH₂ |
| Trp | D-Arg | Phe | Lys | NH₂ |
| Trp | D-Arg | Tyr | Lys | NH₂ |
| Trp | D-Arg | Trp | Lys | NH₂ |
| Trp | D-Arg | Dmt | Lys | NH₂ |
| D-Arg | Trp | Lys | Phe | NH₂ |
| D-Arg | Trp | Phe | Lys | NH₂ |
| D-Arg | Trp | Lys | Dmt | NH₂ |
| D-Arg | Trp | Dmt | Lys | NH₂ |
| D-Arg | Lys | Trp | Phe | NH₂ |
| D-Arg | Lys | Trp | Dmt | NH₂ |
| Cha | D-Arg | Phe | Lys | NH₂ |
| Ala | D-Arg | Phe | Lys | NH₂ |

Cha = cyclohexyl alanine

The amino acids of the peptides in the tables may be in either the L- or the D-configuration unless otherwise indicated

TABLE 4

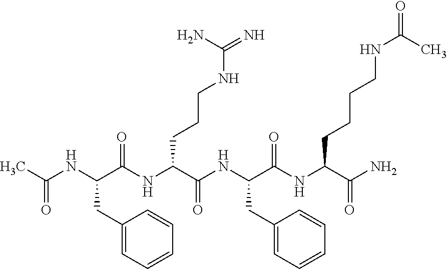 [SMILES: CC(=O)NCCCC[C@H](NC(=O)[C@H](Cc1ccccc1)NC(=O)[C@@H](CCCNC(=N)N)NC(=O)[C@H](Cc1ccccc1)NC(C)=O)C(N)=O]

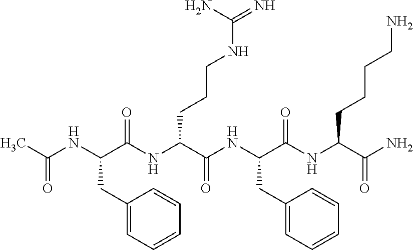 [SMILES: CC(=O)N[C@@H](Cc1ccccc1)C(=O)N[C@H](CCCNC(=N)N)C(=O)N[C@@H](Cc1ccccc1)C(=O)N[C@@H](CCCCN)C(N)=O]

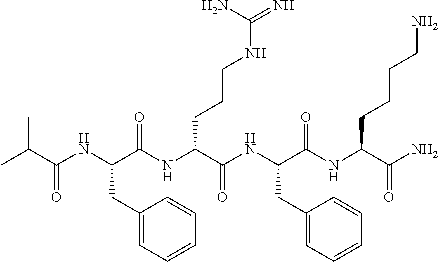 [SMILES: CC(C)C(=O)N[C@@H](Cc1ccccc1)C(=O)N[C@H](CCCNC(=N)N)C(=O)N[C@@H](Cc1ccccc1)C(=O)N[C@@H](CCCCN)C(N)=O]

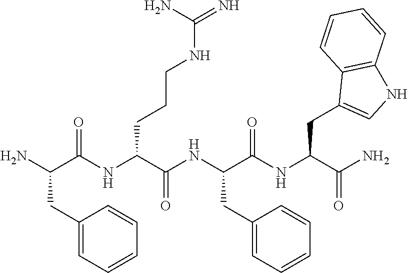 [SMILES: N=C(N)NCCC[C@@H](NC(=O)[C@@H](N)Cc1ccccc1)C(=O)N[C@@H](Cc1ccccc1)C(=O)N[C@@H](Cc1c[nH]c2ccccc12)C(N)=O]

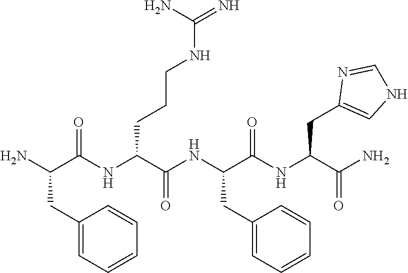 [SMILES: N=C(N)NCCC[C@@H](NC(=O)[C@@H](N)Cc1ccccc1)C(=O)N[C@@H](Cc1ccccc1)C(=O)N[C@@H](Cc1c[nH]cn1)C(N)=O]

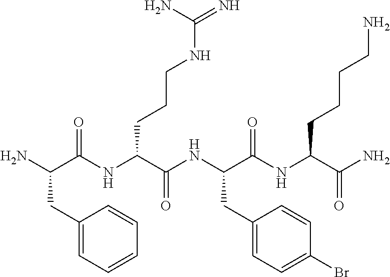 [SMILES: N=C(N)NCCC[C@@H](NC(=O)[C@@H](N)Cc1ccccc1)C(=O)N[C@@H](Cc1ccc(Br)cc1)C(=O)N[C@@H](CCCCN)C(N)=O]

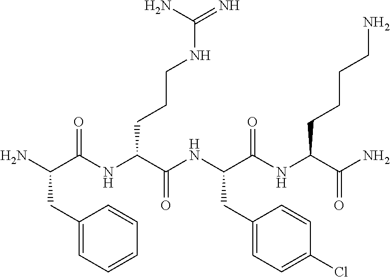 [SMILES: N=C(N)NCCC[C@@H](NC(=O)[C@@H](N)Cc1ccccc1)C(=O)N[C@@H](Cc1ccc(Cl)cc1)C(=O)N[C@@H](CCCCN)C(N)=O]

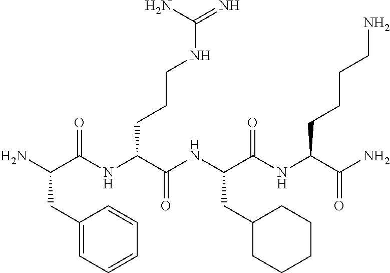 [SMILES: N=C(N)NCCC[C@@H](NC(=O)[C@@H](N)Cc1ccccc1)C(=O)N[C@@H](CC1CCCCC1)C(=O)N[C@@H](CCCCN)C(N)=O]

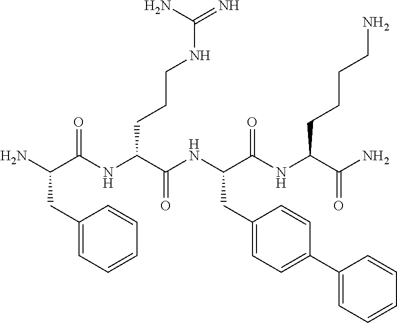 [SMILES: N=C(N)NCCC[C@@H](NC(=O)[C@@H](N)Cc1ccccc1)C(=O)N[C@@H](Cc1ccc(-c2ccccc2)cc1)C(=O)N[C@@H](CCCCN)C(N)=O]

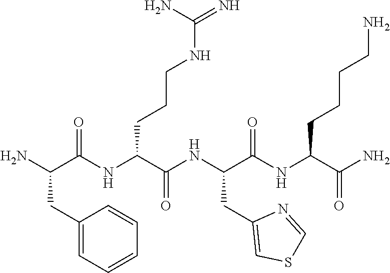 [SMILES: N=C(N)NCCC[C@@H](NC(=O)[C@@H](N)Cc1ccccc1)C(=O)N[C@@H](Cc1cscn1)C(=O)N[C@@H](CCCCN)C(N)=O]

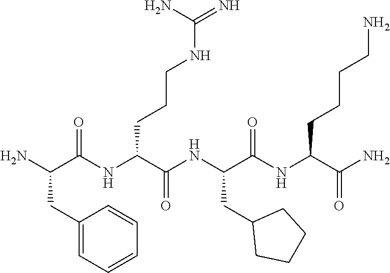 [SMILES: N=C(N)NCCC[C@@H](NC(=O)[C@@H](N)Cc1ccccc1)C(=O)N[C@@H](CC1CCCC1)C(=O)N[C@@H](CCCCN)C(N)=O]

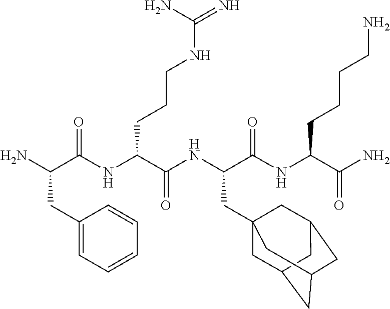 [SMILES: N=C(N)NCCC[C@@H](NC(=O)[C@@H](N)Cc1ccccc1)C(=O)N[C@@H](CC12CC3CC(CC(C3)C1)C2)C(=O)N[C@@H](CCCCN)C(N)=O]

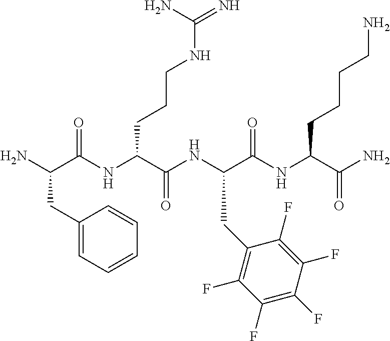 [SMILES: N=C(N)NCCC[C@@H](NC(=O)[C@@H](N)Cc1ccccc1)C(=O)N[C@@H](Cc1c(F)c(F)c(F)c(F)c1F)C(=O)N[C@@H](CCCCN)C(N)=O]

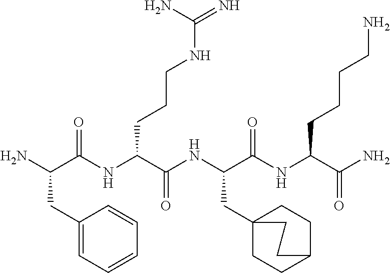 [SMILES: N=C(N)NCCC[C@@H](NC(=O)[C@@H](N)Cc1ccccc1)C(=O)N[C@@H](CC12CCC(CC1)CC2)C(=O)N[C@@H](CCCCN)C(N)=O]

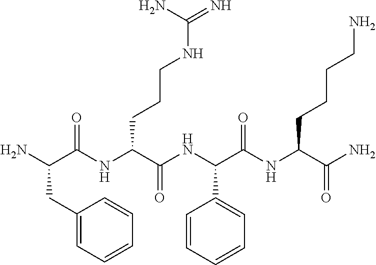 [SMILES: N=C(N)NCCC[C@@H](NC(=O)[C@@H](N)Cc1ccccc1)C(=O)N[C@H](C(=O)N[C@@H](CCCCN)C(N)=O)c1ccccc1]

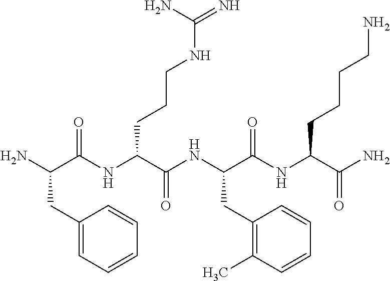 [SMILES: Cc1ccccc1C[C@H](NC(=O)[C@@H](CCCNC(=N)N)NC(=O)[C@@H](N)Cc1ccccc1)C(=O)N[C@@H](CCCCN)C(N)=O]

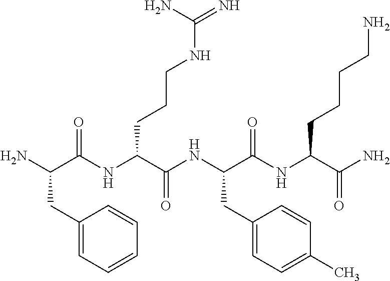 [SMILES: Cc1ccc(C[C@H](NC(=O)[C@@H](CCCNC(=N)N)NC(=O)[C@@H](N)Cc2ccccc2)C(=O)N[C@@H](CCCCN)C(N)=O)cc1]

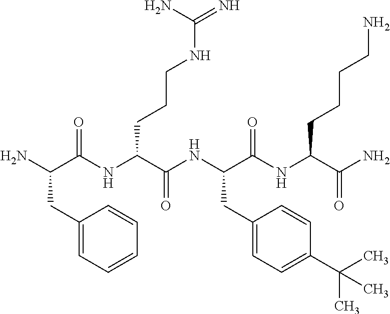 [SMILES: CC(C)(C)c1ccc(C[C@H](NC(=O)[C@@H](CCCNC(=N)N)NC(=O)[C@@H](N)Cc2ccccc2)C(=O)N[C@@H](CCCCN)C(N)=O)cc1]

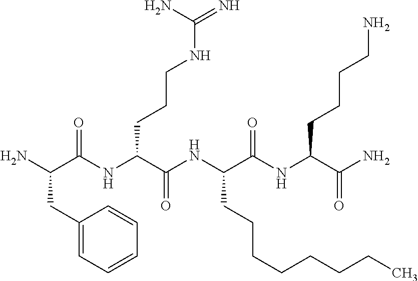 [SMILES: CCCCCCCC[C@H](NC(=O)[C@@H](CCCNC(=N)N)NC(=O)[C@@H](N)Cc1ccccc1)C(=O)N[C@@H](CCCCN)C(N)=O]

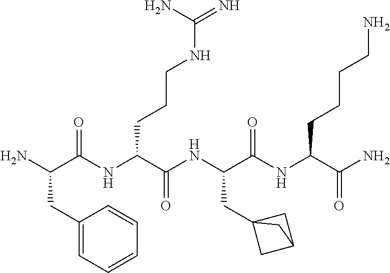 [SMILES: N=C(N)NCCC[C@@H](NC(=O)[C@@H](N)Cc1ccccc1)C(=O)N[C@@H](CC12CC(C1)C2)C(=O)N[C@@H](CCCCN)C(N)=O]

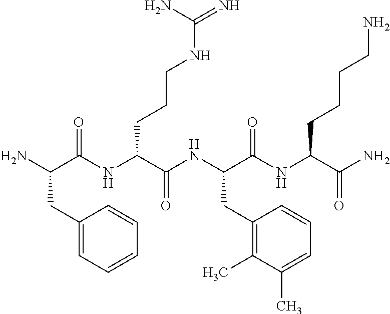 [SMILES: Cc1cccc(C[C@H](NC(=O)[C@@H](CCCNC(=N)N)NC(=O)[C@@H](N)Cc2ccccc2)C(=O)N[C@@H](CCCCN)C(N)=O)c1C]

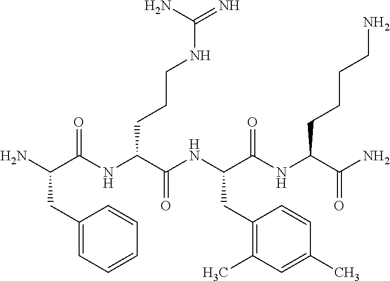 [SMILES: Cc1ccc(C[C@H](NC(=O)[C@@H](CCCNC(=N)N)NC(=O)[C@@H](N)Cc2ccccc2)C(=O)N[C@@H](CCCCN)C(N)=O)c(C)c1]

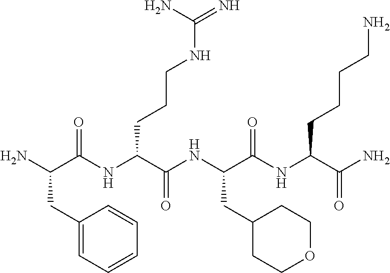 [SMILES: N=C(N)NCCC[C@@H](NC(=O)[C@@H](N)Cc1ccccc1)C(=O)N[C@@H](CC1CCOCC1)C(=O)N[C@@H](CCCCN)C(N)=O]

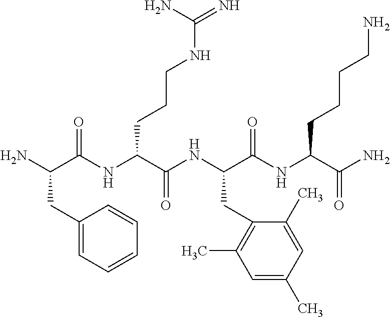 [SMILES: Cc1cc(C)c(C[C@H](NC(=O)[C@@H](CCCNC(=N)N)NC(=O)[C@@H](N)Cc2ccccc2)C(=O)N[C@@H](CCCCN)C(N)=O)c(C)c1]

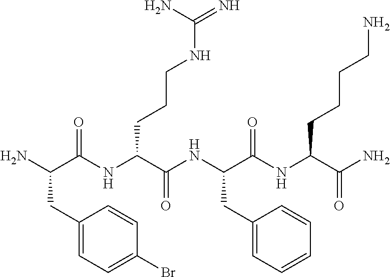 [SMILES: N=C(N)NCCC[C@@H](NC(=O)[C@@H](N)Cc1ccc(Br)cc1)C(=O)N[C@@H](Cc1ccccc1)C(=O)N[C@@H](CCCCN)C(N)=O]

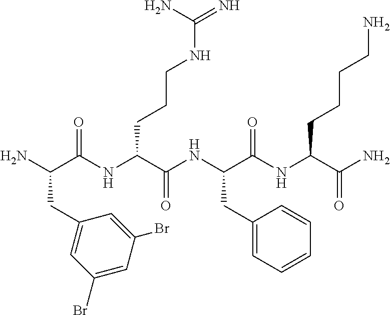 [SMILES: N=C(N)NCCC[C@@H](NC(=O)[C@@H](N)Cc1cc(Br)cc(Br)c1)C(=O)N[C@@H](Cc1ccccc1)C(=O)N[C@@H](CCCCN)C(N)=O]

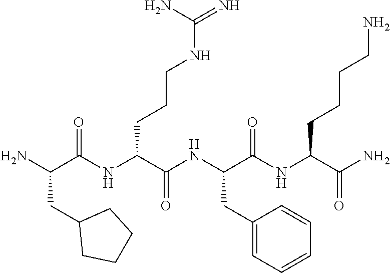 [SMILES: N=C(N)NCCC[C@@H](NC(=O)[C@@H](N)CC1CCCC1)C(=O)N[C@@H](Cc1ccccc1)C(=O)N[C@@H](CCCCN)C(N)=O]

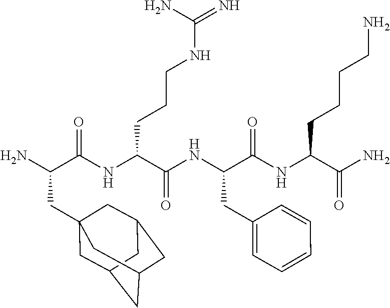 [SMILES: N=C(N)NCCC[C@@H](NC(=O)[C@@H](N)CC12CC3CC(CC(C3)C1)C2)C(=O)N[C@@H](Cc1ccccc1)C(=O)N[C@@H](CCCCN)C(N)=O]

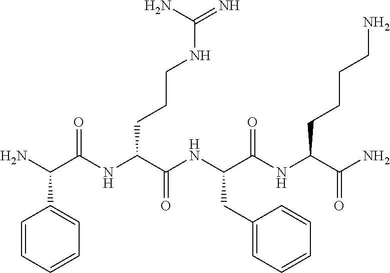 [SMILES: N=C(N)NCCC[C@@H](NC(=O)[C@@H](N)c1ccccc1)C(=O)N[C@@H](Cc1ccccc1)C(=O)N[C@@H](CCCCN)C(N)=O]

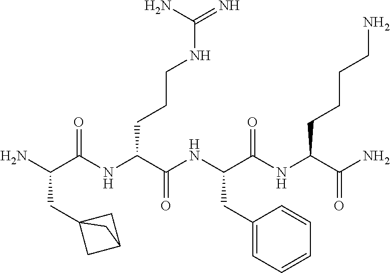 [SMILES: N=C(N)NCCC[C@@H](NC(=O)[C@@H](N)CC12CC(C1)C2)C(=O)N[C@@H](Cc1ccccc1)C(=O)N[C@@H](CCCCN)C(N)=O]

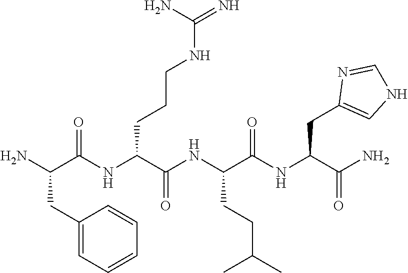 [SMILES: CC(C)CC[C@H](NC(=O)[C@@H](CCCNC(=N)N)NC(=O)[C@@H](N)Cc1ccccc1)C(=O)N[C@@H](Cc1c[nH]cn1)C(N)=O]

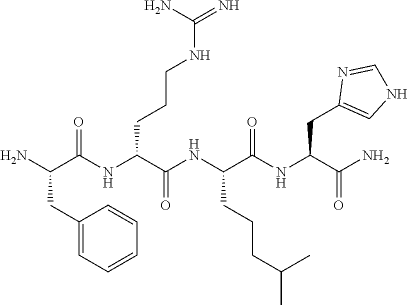 [SMILES: CC(C)CCC[C@H](NC(=O)[C@@H](CCCNC(=N)N)NC(=O)[C@@H](N)Cc1ccccc1)C(=O)N[C@@H](Cc1c[nH]cn1)C(N)=O]

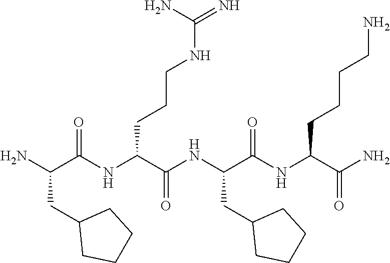 [SMILES: N=C(N)NCCC[C@@H](NC(=O)[C@@H](N)CC1CCCC1)C(=O)N[C@@H](CC1CCCC1)C(=O)N[C@@H](CCCCN)C(N)=O]

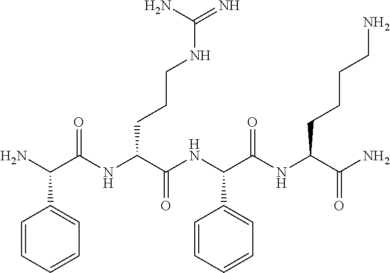 [SMILES: N=C(N)NCCC[C@@H](NC(=O)[C@@H](N)c1ccccc1)C(=O)N[C@H](C(=O)N[C@@H](CCCCN)C(N)=O)c1ccccc1]

TABLE A

| | |
|---|---|
| | 2′,6′-Dmt-D-Arg-Phe-Ahp-NH₂ |
| Tyr-D-Arg-Phe-Lys-NH₂ | 2′,6′-Dmt-D-Arg-Phe-Lys-NH₂ |
| D-Arg-Dmt-Lys-Phe-NH₂ | 2′,6′-Dmt-D-Cit-Phe-Lys-NH₂ |
| D-Arg-Dmt-Phe-Lys-NH₂ | D-Arg-2′,6′-Dmt-Lys-Phe-NH₂ |
| D-Arg-Phe-Lys-Dmt-NH₂ | D-Tyr-Trp-Lys-NH₂ |
| D-Arg-Phe-Dmt-Lys-NH₂ | Lys-D-Arg-Tyr-NH₂ |
| D-Arg-Lys-Dmt-Phe-NH₂ | Met-Tyr-D-Arg-Phe-Arg-NH₂ |
| D-Arg-Lys-Phe-Dmt-NH₂ | Met-Tyr-D-Lys-Phe-Arg |
| D-Arg-Dmt-Lys-Phe-Cys-NH₂ | Phe-Arg-D-His-Asp |
| Phe-Lys-Dmt-D-Arg-NH₂ | Phe-D-Arg-2′,6′-Dmt-Lys-NH₂ |
| Phe-Lys-D-Arg-Dmt-NH₂ | Phe-D-Arg-His |
| Phe-D-Arg-Phe-Lys-NH₂ | Trp-D-Lys-Tyr-Arg-NH₂ |
| Phe-D-Arg-Phe-Lys-Cys-NH₂ | Tyr-D-Arg-Phe-Lys-Glu-NH₂ |
| Phe-D-Arg-Phe-Lys-Ser-Cys-NH₂ | Tyr-His-D-Gly-Met |
| Phe-D-Arg-Phe-Lys-Gly-Cys-NH₂ | D-Arg-Tyr-Lys-Phe-NH₂ |
| Phe-D-Arg-Dmt-Lys-NH₂ | D-Arg-D-Dmt-Lys-Phe-NH₂ |
| Phe-D-Arg-Dmt-Lys-Cys-NH₂ | D-Arg-Dmt-D-Lys-Phe-NH₂ |
| Phe-D-Arg-Dmt-Lys-Ser-Cys-NH₂ | D-Arg-Dmt-Lys-D-Phe-NH₂ |
| Phe-D-Arg-Dmt-Lys-Gly-Cys-NH₂ | D-Arg-D-Dmt-D-Lys-D-Phe-NH₂ |
| Phe-D-Arg-Lys-Dmt-NH₂ | Phe-D-Arg-D-Phe-Lys-NH₂ |
| Phe-Dmt-D-Arg-Lys-NH₂ | Phe-D-Arg-Phe-D-Lys-NH₂ |
| Phe-Dmt-Lys-D-Arg-NH₂ | D-Phe-D-Arg-D-Phe-D-Lys-NH₂ |
| Lys-Phe-D-Arg-Dmt-NH₂ | Lys-D-Phe-Arg-Dmt-NH₂ |
| Lys-Phe-Dmt-D-Arg-NH₂ | D-Arg-Arg-Dmt-Phe-NH₂ |
| Lys-Dmt-D-Arg-Phe-NH₂ | Dmt-D-Phe-Arg-Lys-NH₂ |
| Lys-Dmt-Phe-D-Arg-NH₂ | Phe-D-Dmt-Arg-Lys-NH₂ |
| Lys-D-Arg-Phe-Dmt-NH₂ | D-Arg-Dmt-Lys-NH₂ |
| Lys-D-Arg-Dmt-Phe-NH₂ | Arg-D-Dmt-Lys-NH₂ |
| D-Arg-Dmt-D-Arg-Phe-NH₂ | D-Arg-Dmt-Phe-NH₂ |
| D-Arg-Dmt-D-Arg-Dmt-NH₂ | Arg-D-Dmt-Arg-NH₂ |
| D-Arg-Dmt-D-Arg-Tyr-NH₂ | Dmt-D-Arg-NH₂ |
| D-Arg-Dmt-D-Arg-Trp-NH₂ | D-Arg-Dmt-NH₂ |
| Trp-D-Arg-Tyr-Lys-NH₂ | D-Dmt-Arg-NH₂ |
| Trp-D-Arg-Trp-Lys-NH₂ | Arg-D-Dmt-NH₂ |
| Trp-D-Arg-Dmt-Lys-NH₂ | D-Arg-D-Dmt-NH₂ |
| D-Arg-Trp-Lys-Phe-NH₂ | D-Arg-D-Tyr-Lys-Phe-NH₂ |
| D-Arg-Trp-Phe-Lys-NH₂ | D-Arg-Tyr-D-Lys-Phe-NH₂ |
| D-Arg-Trp-Lys-Dmt-NH₂ | D-Arg-Tyr-Lys-D-Phe-NH₂ |
| D-Arg-Trp-Dmt-Lys-NH₂ | D-Arg-D-Tyr-D-Lys-D-Phe-NH₂ |
| D-Arg-Lys-Trp-Phe-NH₂ | Lys-D-Phe-Arg-Tyr-NH₂ |
| D-Arg-Lys-Trp-Dmt-NH₂ | D-Arg-Arg-Tyr-Phe-NH₂ |
| Cha-D-Arg-Phe-Lys-NH₂ | Tyr-D-Phe-Arg-Lys-NH₂ |
| Ala-D-Arg-Phe-Lys-NH₂ | Phe-D-Tyr-Arg-Lys-NH₂ |
| 2′,6′-Dmp-D-Arg-2′,6′-Dmt-Lys-NH₂ | D-Arg-Tyr-Lys-NH₂ |
| 2′,6′-Dmp-D-Arg-Phe-Lys-NH₂ | Arg-D-Tyr-Lys-NH₂ |
| 2′,6′-Dmt-D-Arg-Phe-Orn-NH₂ | D-Arg-Tyr-Phe-NH₂ |
| Arg-D-Tyr-Arg-NH₂ | Arg-Cha-Lys |
| Tyr-D-Arg-NH₂ | Arg-Dmt |
| D-Arg-Tyr-NH₂ | Arg-Dmt-Arg |
| D-Tyr-Arg-NH₂ | Arg-Dmt-Lys |
| Arg-D-Tyr-NH₂ | Arg-Dmt-Lys-Phe |
| D-Arg-D-Tyr-NH₂ | Arg-Dmt-Lys-Phe-Cys |
| Dmt-Lys-Phe-NH₂ | Arg-Dmt-Phe |
| Lys-Dmt-D-Arg-NH₂ | Arg-Dmt-Phe-Lys |
| Phe-Lys-Dmt-NH₂ | Arg-Lys-Dmt-Phe |
| D-Arg-Phe-Lys-NH₂ | Arg-Lys-Phe-Dmt |
| D-Arg-Cha-Lys-NH₂ | Arg-Phe-Dmt-Lys |
| D-Arg-Trp-Lys-NH₂ | Arg-Phe-Lys |
| Dmt-Lys-D-Phe-NH₂ | Arg-Trp-Lys |
| Dmt-Lys-NH₂ | Arg-Tyr-Lys |
| Lys-Phe-NH₂ | Arg-Tyr-Lys-Phe |
| D-Arg-Cha-Lys-Cha-NH₂ | D-Arg-D-Dmt-D-Lys-L-Phe-NH₂ |
| D-Nle-Dmt-Ahp-Phe-NH₂ | D-Arg-D-Dmt-L-Lys-D-Phe-NH₂ |
| D-Nle-Cha-Ahp-Cha-NH₂ | D-Arg-D-Dmt-L-Lys-L-Phe-NH₂ |
| D-Arg-Dmt-D-Lys-NH₂ | D-Arg-Dmt-D-Lys-NH₂ |
| D-Arg-Dmt-D-Lys-Phe-NH₂ | D-Arg-Dmt-Lys-NH₂ |
| Lys-Trp-D-Arg-NH₂ | D-Arg-Dmt-Lys-Phe-Cys |
| H-Lys-D-Phe-Arg-Dmt-NH₂ | D-Arg-L-Dmt-D-Lys-D-Phe-NH₂ |
| H-D-Arg-Lys-Dmt-Phe-NH₂ | D-Arg-L-Dmt-D-Lys-L-Phe-NH₂ |
| H-D-Arg-Lys-Phe-Dmt-NH₂ | D-Arg-L-Dmt-L-Lys-D-Phe-NH₂ |
| H-D-Arg Arg-Dmt-Phe-NH₂ | Dmt-Arg |
| H-D-Arg-Dmt-Phe-Lys-NH₂ | Dmt-Lys |
| H-D-Arg-Phe-Dmt-Lys-NH₂ | Dmt-Lys-Phe |
| H-Dmt-D-Phe-Arg-Lys-NH₂ | Dmt-Phe-Arg-Lys |
| H-Phe-D-Dmt-Arg-Lys-NH₂ | H-Arg-D-Dmt-Lys-Phe-NH₂ |
| H-D-Arg-Dmt-Lys-NH₂ | H-Arg-Dmt-Lys-Phe-NH₂ |
| H-D-Arg-Dmt-D-Lys-D-Phe-NH₂ | H-D-Arg-2,6-dichloro-L-tyrosine-L-Lys- |
| | L-Phe-NH₂ |
| H-D-Arg-D-Dmt-Lys-Phe-NH₂ | H-D-Arg-2,6-dichlorotyrosine-Lys-Phe-NH₂ |
| H-D-Arg-Dmt-Phe-NH₂ | H-D-Arg-2,6-difluoro-L-tyrosine-L-Lys- |
| | L-Phe-NH₂ |
| H-Dmt-D-Arg-NH₂ | H-D-Arg-2,6,difluorotyrosine-Lys-Phe-NH₂ |
| H-Phe-D-Arg-D-Phe-Lys-NH₂ | H-D-Arg-2,6-dimethyl-L-phenylalanine-L- |
| | Lys-L-Phe-NH₂ |
| H-Phe-D-Arg-Phe-D-Lys-NH₂ | H-D-Arg-2,6-dimethylphenylalanine- |
| | Lys-Phe-NH₂ |
| H-D-Phe-D-Arg-D-Phe-D-Lys-NH₂ | H-D-Arg-4-methoxy-2,6-dimethyl-L- |
| | phenylalanine-L-Lys-L-Phe-NH₂ |
| H-D-Arg-D-Dmt-D-Lys-D-Phe-NH₂ | H-D-Arg-4-methoxy-2,6- |
| | dimethylphenylalanine-Lys-Phe-NH₂ |
| H-D-Arg-Cha-Lys-NH₂ | H-Arg-D-Dmt-Lys-NH₂ |
| H-D-Arg-Cha-Lys-Cha-NH₂ | H-Arg-D-Dmt-Arg-NH₂ |
| H-D-Dmt-Arg-NH₂ | H-D-Arg-Tyr-Lys-Phe-NH₂ |
| H-Arg-D-Dmt-NH₂ | H-D-His-L-Dmt-L-Lys-L-Phe-NH₂ |
| H-D-Arg-D-Dmt-NH₂ | H-D-Lys-L-Dmt-L-Lys-L-Phe-NH₂ |
| Arg-Arg-Dmt-Phe | H-Dmt-D-Arg-Lys-Phe-NH₂ |
| H-D-Arg-Dmt-Lys-2,6- | H-Dmt-D-Arg-Phe-Lys-NH₂ |
| dimethylphenylalanine-NH₂ | |
| H-D-Arg-Dmt-Lys-3- | H-Dmt-Lys-D-Arg-Phe-NH₂ |
| hydroxyphenylalanine-NH₂ | |
| H-D-Arg-Dmt-N6-acetyllysine-Phe-NH₂ | H-Dmt-Lys-Phe-D-Arg-NH₂ |
| H-D-Arg-D-Phe-L-Lys-L-Phe-NH₂ | H-Dmt-Phe-D-Arg-Lys-NH₂ |
| H-D-Arg-D-Trp-L-Lys-L-Phe-NH₂ | H-Dmt-Phe-Lys-D-Arg-NH₂ |
| H-D-Arg-D-Tyr-L-Lys-L-Phe-NH₂ | H-L-Dmt-D-Arg-L-Lys-L-Phe-NH₂ |
| H-D-Arg-L-Dmt-L-Lys-2,6-dimethyl- | H-L-Dmt-D-Arg-L-Phe-L-Lys-NH₂ |
| L-phenylalanine-NH₂ | |
| H-D-Arg-L-Dmt-L-Lys-3-hydroxy-L- | H-L-Dmt-L-Lys-D-Arg-L-Phe-NH₂ |
| phenylalanine-NH2 | |
| H-D-Arg-L-Dmt-L-Lys-D-Dmt-NH₂ | H-L-Dmt-L-Lys-L-Phe-D-Arg-NH₂ |
| H-D-Arg-L-Dmt-L-Lys-D-Trp-NH₂ | H-L-Dmt-L-Phe-D-Arg-L-Lys-NH₂ |
| H-D-Arg-L-Dmt-L-Lys-D-Tyr-NH₂ | H-L-Dmt-L-Phe-L-Lys-D-Arg-NH₂ |
| H-D-Arg-L-Dmt-L-Lys-L-Dmt-NH₂ | H-L-His-L-Dmt-L-Lys-L-Phe-NH₂ |
| H-D-Arg-L-Dmt-L-Lys-L-Trp-NH₂ | H-L-Lys-D-Arg-L-Dmt-L-Phe-NH₂ |
| H-D-Arg-L-Dmt-L-Lys-L-Tyr-NH₂ | H-L-Lys-D-Arg-L-Phe-L-Dmt-NH₂ |
| H-D-Arg-L-Dmt-L-Phe-L-Lys-NH₂ | H-L-Lys-L-Dmt-D-Arg-L-Phe-NH₂ |
| H-D-Arg-L-Dmt-N6-acetyl-L-lysine- | H-L-Lys-L-Dmt-L-Lys-L-Phe-NH₂ |
| L-Phe-NH₂ | |
| H-D-Arg-L-Lys-L-Dmt-L-Phe-NH₂ | H-L-Lys-L-Dmt-L-Phe-D-Arg-NH₂ |
| H-D-Arg-L-Lys-L-Phe-L-Dmt-NH₂ | H-L-Lys-L-Phe-D-Arg-L-Dmt-NH₂ |
| H-D-Arg-L-Phe-L-Dmt-L-Lys-NH₂ | H-L-Lys-L-Phe-L-Dmt-D-Arg-NH₂ |
| H-D-Arg-L-Phe-L-Lys-L-Dmt-NH₂ | H-L-Phe-D-Arg-L-Dmt-L-Lys-NH₂ |
| H-D-Arg-L-Phe-L-Lys-L-Phe-NH₂ | H-L-Phe-D-Arg-L-Lys-L-Dmt-NH₂ |
| H-D-Arg-L-Trp-L-Lys-L-Phe-NH₂ | H-L-Phe-L-Dmt-D-Arg-L-Lys-NH₂ |
| H-D-Arg-L-Tyr-L-Lys-L-Phe-NH₂ | H-L-Phe-L-Dmt-L-Lys-D-Arg-NH₂ |
| H-D-Arg-Phe-Lys-Dmt-NH₂ | H-L-Phe-L-Lys-D-Arg-L-Dmt-NH₂ |
| H-D-Arg-Tyr-Lys-Phe-NH₂ | H-L-Phe-L-Lys-L-Dmt-D-Arg-NH₂ |
| H-D-His-L-Dmt-L-Lys-L-Phe-NH₂ | H-Lys-D-Arg-Dmt-Phe-NH₂ |
| H-D-Lys-L-Dmt-L-Lys-L-Phe-NH₂ | H-Lys-D-Arg-Phe-Dmt-NH₂ |
| H-Dmt-D-Arg-Lys-Phe-NH₂ | H-Lys-Dmt-D-Arg-Phe-NH₂ |
| H-Dmt-D-Arg-Phe-Lys-NH₂ | H-Lys-Dmt-Phe-D-Arg-NH₂ |
| H-Dmt-Lys-D-Arg-Phe-NH₂ | H-Lys-Phe-D-Arg-Dmt-NH₂ |
| H-Dmt-Lys-Phe-D-Arg-NH₂ | H-Lys-Phe-Dmt-D-Arg-NH₂ |
| H-Dmt-Phe-D-Arg-Lys-NH₂ | H-Phe-Arg-Phe-Lys-NH₂ |
| H-D-Arg-Tyr-Lys-Phe-NH₂ | H-Phe-D-Arg-Dmt-Lys-NH₂ |
| H-D-His-L-Dmt-L-Lys-L-Phe-NH₂ | H-Phe-D-Arg-Lys-Dmt-NH₂ |
| H-D-Lys-L-Dmt-L-Lys-L-Phe-NH₂ | H-Phe-Dmt-D-Arg-Lys-NH₂ |
| H-Dmt-D-Arg-Lys-Phe-NH₂ | H-Phe-Dmt-Lys-D-Arg-NH₂ |
| H-Dmt-D-Arg-Phe-Lys-NH₂ | H-Phe-Lys-D-Arg-Dmt-NH₂ |
| H-Phe-Lys-Dmt-D-Arg-NH₂ | H-D-Arg-Dmt-Lys-Phe-OH |
| L-Arg-D-Dmt-D-Lys-D-Phe-NH₂ | |
| L-Arg-D-Dmt-D-Lys-L-Phe-NH₂ | |
| L-Arg-D-Dmt-L-Lys-D-Phe-NH₂ | |
| L-Arg-D-Dmt-L-Lys-L-Phe-NH₂ | |
| L-Arg-L-Dmt-D-Lys-D-Phe-NH₂ | |
| L-Arg-L-Dmt-D-Lys-L-Phe-NH₂ | |
| L-Arg-L-Dmt-L-Lys-D-Phe-NH₂ | |
| L-Arg-L-Dmt-L-Lys-L-Phe-NH₂ | |
| Lys-Dmt-Arg | |
| Lys-Phe | |
| Lys-Phe-Arg-Dmt | |
| Lys-Trp-Arg | |
| Phe-Arg-Dmt-Lys | |
| Phe-Arg-Phe-Lys | |
| Phe-Dmt-Arg-Lys | |
| Phe-Lys-Dmt | |
| Arg-Dmt-Lys-Phe-NH₂ | |
| Phe-Dmt-Arg-Lys-NH₂ | |
| Phe-Lys-Dmt-Arg-NH₂ | |
| Dmt-Arg-Lys-Phe-NH₂ | |
| Lys-Dmt-Arg-Phe-NH₂ | |
| Phe-Dmt-Lys-Arg-NH₂ | |
| Arg-Lys-Dmt-Phe-NH₂ | |
| Arg-Dmt-Phe-Lys-NH₂ | |
| D-Arg-Dmt-Lys-Phe-NH₂ | |
| Dmt-D-Arg-Phe-Lys-NH₂ | |
| H-Phe-D-Arg-Phe-Lys-Cys-NH₂ | |
| D-Arg-Dmt-Lys-Trp-NH₂ | |
| D-Arg-Trp-Lys-Trp-NH₂ | |
| H-D-Arg-Dmt-Lys-Phe(NMe)-NH₂ | |
| H-D-Arg-Dmt-Lys(N^{α}Me)-Phe(NMe)-NH₂ | |
| H-D-Arg(N^{α}Me)-Dmt(NMe)-Lys(N^{α}Me)- | |
| Phe(NMe)-NH₂ | |
| D-Arg-2′6′Dmt-Lys-Phe-NH₂ | |
| H-Phe-D-Arg-Phe-Lys-Cys-NH₂ | |
| D-Arg-Dmt-Lys-Phe-Ser-Cys-NH₂ | |
| D-Arg-Dmt-Lys-Phe-Gly-Cys-NH₂ | |
| Gly-D-Phe-Lys-His-D-Arg-Tyr-NH₂ | |
| D-Arg-Dmt-Lys-Phe-Met-NH₂ | |
| D-Arg-Dmt-Lys-Phe-Lys-Trp-NH₂ | |
| D-Arg-Dmt-Lys-Dmt-Lys-Trp-NH₂ | |
| D-Arg-Dmt-Lys-Phe-Lys-Met-NH₂ | |
| D-Arg-Dmt-Lys-Dmt-Lys-Met-NH₂ | |
| H-D-Arg-Dmt-Lys-OH | |
| H-D-Arg-Dmt-OH | |

TABLE B

| Amino | Amino | Amino | Amino | |
|---|---|---|---|---|
| Acid | Acid | Acid | Acid | C-Terminal |
| Position 1 | Position 2 | Position 3 | Position 4 | Modification |
| Tyr | D-Arg | Phe | Orn | NH₂ |
| Tyr | D-Arg | Phe | Dab | NH₂ |
| Tyr | D-Arg | Phe | Dap | NH₂ |
| 2′6′Dmt | D-Arg | Phe | Lys-NH(CH₂)₂- | NH₂ |
| | | | NH-dns | |
| 2′6′Dmt | D-Arg | Phe | Lys-NH(CH₂)₂- | NH₂ |
| | | | NH-atm | |
| 2′6′Dmt | D-Arg | Phe | dnsLys | NH₂ |
| 2′6′Dmt | D-Cit | Phe | Ahp | NH₂ |
| 2′6′Dmt | D-Arg | Phe | Dab | NH₂ |
| 2′6′Dmt | D-Arg | Phe | Dap | NH₂ |
| 3′5′Dmt | D-Arg | Phe | Lys | NH₂ |
| 3′5′Dmt | D-Arg | Phe | Orn | NH₂ |
| 3′5′Dmt | D-Arg | Phe | Dab | NH₂ |
| 3′5′Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′6′Dmt | D-Arg | Tyr | Lys | NH₂ |
| 2′6′Dmt | D-Arg | Tyr | Orn | NH₂ |
| 2′6′Dmt | D-Arg | Tyr | Dab | NH₂ |
| 2′6′Dmt | D-Arg | Tyr | Dap | NH₂ |
| 2′6′Dmt | D-Arg | 2′6′Dmt | Lys | NH₂ |
| 2′6′Dmt | D-Arg | 2′6′Dmt | Orn | NH₂ |
| 2′6′Dmt | D-Arg | 2′6′Dmt | Dab | NH₂ |
| 2′6′Dmt | D-Arg | 2′6′Dmt | Dap | NH₂ |
| 3′5′Dmt | D-Arg | 3′5′Dmt | Arg | NH₂ |
| 3′5′Dmt | D-Arg | 3′5′Dmt | Lys | NH₂ |
| 3′5′Dmt | D-Arg | 3′5′Dmt | Orn | NH₂ |
| 3′5′Dmt | D-Arg | 3′5′Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′6′Dmt | D-Lys | Phe | Dab | NH₂ |
| 2′6′Dmt | D-Lys | Phe | Dap | NH₂ |
| 2′6′Dmt | D-Lys | Phe | Arg | NH₂ |
| 2′6′Dmt | D-Lys | Phe | Lys | NH₂ |
| 3′5′Dmt | D-Lys | Phe | Orn | NH₂ |
| 3′5′Dmt | D-Lys | Phe | Dab | NH₂ |
| 3′5′Dmt | D-Lys | Phe | Dap | NH₂ |
| 3′5′Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′6′Dmt | D-Lys | Tyr | Lys | NH₂ |
| 2′6′Dmt | D-Lys | Tyr | Orn | NH₂ |
| 2′6′Dmt | D-Lys | Tyr | Dab | NH₂ |
| 2′6′Dmt | D-Lys | Tyr | Dap | NH₂ |
| 2′6′Dmt | D-Lys | 2′6′Dmt | Lys | NH₂ |
| 2′6′Dmt | D-Lys | 2′6′Dmt | Orn | NH₂ |
| 2′6′Dmt | D-Lys | 2′6′Dmt | Dab | NH₂ |
| 2′6′Dmt | D-Lys | 2′6′Dmt | Dap | NH₂ |
| 3′5′Dmt | D-Lys | 3′5′Dmt | Lys | NH₂ |
| 3′5′Dmt | D-Lys | 3′5′Dmt | Orn | NH₂ |
| 3′5′Dmt | D-Lys | 3′5′Dmt | Dab | NH₂ |
| 3′5′Dmt | D-Lys | 3′5′Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′6′Dmt | D-Arg | Phe | Arg | NH₂ |
| 2′6′Dmt | D-Lys | Phe | Arg | NH₂ |
| 2′6′Dmt | D-Orn | Phe | Arg | NH₂ |
| 2′6′Dmt | D-Dab | Phe | Arg | NH₂ |
| 3′5′Dmt | D-Dap | Phe | Arg | NH₂ |
| 3′5′Dmt | D-Arg | Phe | Arg | NH₂ |
| 3′5′Dmt | D-Lys | Phe | Arg | NH₂ |
| 3′5′Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′6′Dmt | D-Arg | 2′6′Dmt | Arg | NH₂ |
| 2′6′Dmt | D-Lys | 2′6′Dmt | Arg | NH₂ |
| 2′6′Dmt | D-Orn | 2′6′Dmt | Arg | NH₂ |
| 2′6′Dmt | D-Dab | 2′6′Dmt | Arg | NH₂ |
| 3′5′Dmt | D-Dap | 3′5′Dmt | Arg | NH₂ |
| 3′5′Dmt | D-Arg | 3′5′Dmt | Arg | NH₂ |
| 3′5′Dmt | D-Lys | 3′5′Dmt | Arg | NH₂ |
| 3′5′Dmt | D-Orn | 3′5′Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |
| Trp | D-Arg | Phe | Lys | NH₂ |

TABLE C

| | |
|---|---|
| D-Arg-Dmt-Lys-Phe-Glu-Cys-Gly-NH₂ | Thr-Gly-Tyr-Arg-D-His-Phe-Trp-D-His-Lys |
| Phe-D-Arg-Phe-Lys-Glu-Cys-Gly-NH₂ | Trp-Lys-Phe-D-Asp-Arg-Tyr-D-His-Lys |
| Phe-D-Arg-Dmt-Lys-Glu-Cys-Gly-NH₂ | Val-D-Lys-His-Tyr-D-Phe-Ser-Tyr-Arg-NH₂ |
| Ala-D-Phe-D-Arg-Tyr-Lys-D-Trp-His-D- | Gly-D-Phe-Lys-Tyr-His-D-Arg-Tyr-NH₂ |
| Tyr-Gly-Phe | |
| Asp-D-Trp-Lys-Tyr-D-His-Phe-Arg-D-Gly- | Asp-D-Trp-Lys-Tyr-D-His-Phe-Arg-D-Gly-Lys- |
| Lys-NH₂ | NH₂ |
| D-His-Glu-Lys-Tyr-D-Phe-Arg | D-His-Lys-Tyr-D-Phe-Glu-D-Asp-D-His-D-Lys- |
| | Arg-Trp-NH₂ |
| D-His-Lys-Tyr-D-Phe-Glu-D-Asp-D-Asp-D- | H-Phe-D-Arg-Phe-Lys-Glu-Cys-Gly-NH₂ |
| His-D-Lys-Arg-Trp-NH₂ | |
| Lys-D-Gln-Tyr-Arg-D-Phe-Trp-NH₂ | Phe-Arg-Phe-Lys-Glu-Cys-Gly |
| Lys-Trp-D-Tyr-Arg-Asn-Phe-Tyr-D-His- | H-D-Arg-Dmt-Lys-Phe-Ser-Gly-Cys-NH₂ |
| NH₂ | |
| Phe-D-Arg-Lys-Trp-Tyr-D-Arg-His | |

TABLE D

| | |
|---|---|
| 6-Butyric acid CoQ0-Phe-D-Arg-Phe-Lys- | Dmt-D-Arg-Ald-Lys-NH₂ |
| NH₂ | |
| 6-Decanoic acid CoQ0-Phe-D-Arg-Phe- | Dmt-D-Arg-Phe-Lys-Ald-NH₂ |
| Lys-NH₂ | |
| H-D-N2-acetylarginine-Dmt-Lys-Phe-NH₂ | Bio-2′6′Dmt-D-Arg-Phe-Lys-NH₂ |
| H-D-N8-acetylarginine-Dmt-Lys-Phe-NH₂ | 2′6′Dmt-D-Arg-Phe-dnsDap-NH₂ |
| H-N2-acetyl-D-arginine-L-Dmt-L-Lys-L- | 2′6′Dmt-D-Arg-Phe-atnDap-NH₂ |
| Phe-NH₂ | |
| H-N7-acetyl-D-arginine-Dmt-Lys-Phe-- | H-D-Arg-Ψ[CH₂—NH]Dmt-Lys-Phe-NH₂ |
| NH₂ | |
| H-Phe(d5)-D-Arg-Phe(d5)-Lys-NH₂ | H-D-Arg-Dmt-Ψ[CH₂—NH]Lys-Phe-NH₂ |
| Succinic monoester CoQ0-Phe-D-Arg- | H-D-Arg-Dmt-LysΨ[CH₂—NH]Phe-NH₂ |
| Phe-Lys-NH₂ | |
| Dmt-D-Arg-Phe-(atn)Dap-NH₂ | H-D-Arg-Dmt-Ψ[CH₂—NH]Lys-Ψ[CH₂—NH]Phe- |
| | NH₂ |
| Dmt-D-Arg-Phe-(dns)Dap-NH₂ | |

TABLE E

| | |
|---|---|
| Arg-D-Leu-D-Tyr-Phe-Lys-Glu-D-Lys-Arg- | Tyr-Asp-D-Lys-Tyr-Phe-D-Lys-D-Arg-Phe-Pro-D- |
| D-Trp-Lys-D-Phe-Tyr-D-Arg-Gly | Tyr-His-Lys |
| Asp-Arg-D-Phe-Cys-Phe-D-Arg-D-Lys-Tyr- | Tyr-D-His-Phe-D-Arg-Asp-Lys-D-Arg-His-Trp-D- |
| Arg-D-Tyr-Trp-D-His-Tyr-D-Phe-Lys-Phe | His-Phe |
| D-Glu-Asp-Lys-D-Arg-D-His-Phe-Phe-D- | Phe-Tyr-Lys-D-Arg-Trp-His-D-Lys-D-Lys-Glu- |
| Val-Tyr-Arg-Tyr-D-Tyr-Arg-His-Phe-NH₂ | Arg-D-Tyr-Thr |
| Glu-Arg-D-Lys-Tyr-D-Val-Phe-D-His-Trp- | Tyr-Asp-D-Lys-Tyr-Phe-D-Lys-D-Arg-Phe-Pro-D- |
| Arg-D-Gly-Tyr-Arg-D-Met-NH₂ | Tyr-His-Lys |
| Gly-Ala-Lys-Phe-D-Lys-Glu-Arg-Tyr-His-D- | Glu-Arg-D-Lys-Tyr-D-Val-Phe-D-His-Trp-Arg-D- |
| Arg-D-Arg-Asp-Tyr-Trp-D-His-Trp-His-D- | Gly-Tyr-Arg-D-Met-NH₂ |
| Lys-Asp | |
| His-Tyr-D-Arg-Trp-Lys-Phe-D-Asp-Ala- | Arg-D-Leu-D-Tyr-Phe-Lys-Glu-D-Lys-Arg-D-Trp- |
| Arg-Cys-D-Tyr-His-Phe-D-Lys-Tyr-His-Ser- | Lys-D-Phe-Tyr-D-Arg-Gly |
| NH₂ | |
| Phe-Phe-D-Tyr-Arg-Glu-Asp-D-Lys-Arg-D- | Gly-Ala-Lys-Phe-D-Lys-Glu-Arg-Tyr-His-D-Arg- |
| Arg-His-Phe-NH₂ | D-Arg-Asp-Tyr-Trp-D-His-Trp-His-D-Lys-Asp |
| Phe-Tyr-Lys-D-Arg-Trp-His-D-Lys-D-Lys- | Gly-Ala-Lys-Phe-D-Lys-Glu-Arg-Tyr-His-D-Arg- |
| Glu-Arg-D-Tyr-Thr | D-Arg-Asp-Tyr-Trp-D-His-Trp-His-D-Lys-Asp |
| Thr-Tyr-Arg-D-Lys-Trp-Tyr-Glu-Asp-D- | |
| Lys-D-Arg-His-Phe-D-Tyr-Gly-Val-Ile-D- | |
| His-Arg-Tyr-Lys-NH₂ | |

Certain embodiments are within the scope of the following numbered paragraphs.

1. A method for reducing or preventing myocardial damage in a subject caused by extracorporeal membrane oxygenation (ECMO), the method comprising administering a cardioprotective agent to the subject and treating the subject with ECMO.

2. A method of reducing or preventing myocardial damage in a subject caused by ECMO, the method comprising treating the subject with normoxemic ECMO.

3. A method for reducing or preventing myocardial damage in a subject caused by ECMO, the method comprising treating the subject with left ventricular (LV) decompression prior to ECMO treatment.

4. The method of any one of paragraphs 1 to 3, (i) the myocardial damage is caused by reduction or depletion of tafazzin levels in cardiac muscle cells of the subject, and the method restores or maintains said tafazzin levels; (ii) the myocardial damage is caused by reduction or depletion of cardiolipin levels in cardiac muscle cells of the subject, and the method restores or maintains said cardiolipin levels; (iii) the myocardial damage is caused by increased iAAA protease (e.g., YME1) activity in cardiac muscle cells of the subject, and the method decreases iAAA protease levels, in order to restore or maintain tafazzin levels; and/or (iv) the mitochondrial protective agent stabilizes cardiolipin, protects tafazzin from ECMO-induced depletion or reduction, or targets a protease that causes tafazzin degradation.

5. A method of reducing, preventing, or treating myocardial damage in a subject caused by ECMO, the method comprising restoring or maintaining tafazzin levels in cardiac muscle cells of the subject by enzyme replacement, gene therapy, CRISPR, mRNA therapy, protease inhibitors, or siRNA, which is directed to tafazzin directly, cardiolipin, and/or an iAAA protease (e.g., YME1).

6. The method of paragraph 5, further comprising treating the subject with ECMO, optionally with preemptive LV decompression.

7. The method of any one of paragraphs 2 to 6, further comprising administering a cardioprotective agent to the subject.

8. The method of paragraph 1 or 7, wherein the cardioprotective agent is administered before, during, or after the ECMO treatment, or in any combination thereof.

9. The method of any one of paragraphs 1 to 8, wherein the subject is being treated for ischemic heart disease.

10. The method of paragraph 9, wherein the ischemic heart disease is selected from acute myocardial infarction, heart failure, shock, and high risk percutaneous coronary intervention (PCI).

11. The method of any one of paragraphs 1 to 10, further comprising treating the subject with coronary artery reperfusion.

12. The method of paragraph 11, wherein, in instances of treatment with a cardioprotective agent, the cardioprotective agent is administered before the start of coronary artery reperfusion.

13. The method of paragraph 12, wherein the cardioprotective agent is administered at least 30 minutes before the start of coronary artery reperfusion.

14. The method of any one of paragraphs 1 to 13, wherein the myocardial damage comprises a myocardial infarction or an increase in the size of an already existing myocardial infarction.

15. The method of any one of paragraphs 1 to 14, wherein the myocardial damage comprises left ventricular (LV) injury.

16. The method of any one of paragraphs 1 to 15, wherein the myocardial damage is characterized by oxidative stress.

17. The method of any one of paragraphs 11 to 16, wherein the myocardial damage is ischemia-reperfusion injury.

18. The method of any one of paragraphs 1 to 17, wherein the subject has or is at risk of developing myocardial infarction, heart failure, cardiac arrest, heart muscle disease, myocarditis, sepsis, hypothermia, post-transplant complications, cardiogenic shock, cardio-respiratory failure, respiratory failure, lung infection, acute respiratory distress syndrome, pulmonary embolism, congenital diaphragmatic hernia, influenza, pulmonary hypertension, pneumonia, respiratory failure, trauma, or Covid-19, or is subject to treatment with or by a ventricular assist device, heart transplant, lung transplant, heart surgery, or cardiac catheterization.

19. The method of any one of paragraphs 1 and 7 to 18 wherein, in instances of treatment with a cardioprotective agent, the cardioprotective agent is administered by an intra-arterial, intra-coronary, intra-myocardial, intra-epicardial, pericardial, or intravenous route, or via the ECMO circuit.

20. The method of any one of paragraphs 1 and 7 to 19, further comprising intravenous administration of a cardioprotective agent.

21. The method of any one of paragraphs 11 to 20, wherein, in instances of treatment with coronary artery reperfusion, ECMO treatment is maintained during the coronary artery reperfusion.

22. The method of any one of paragraphs 1 to 21, wherein the ECMO is veno-arterial ECMO or veno-venous ECMO.

23. The method of paragraph 22, wherein the VA-ECMO is peripheral VA-ECMO.

24. The method of any one of paragraphs 1 and 3 to 23, wherein the ECMO is normoxemic ECMO.

25. The method of any one of paragraphs 1, 2, and 4 to 24, further comprising LV decompression before initiation of ECMO.

26. The method of paragraph 3 or 25, wherein the LV decompression is carried out at least about 30 minutes before initiation of ECMO.

27. The method of paragraph 3, 25, or 26, wherein the LV decompression is carried out using a trans-valvular pump (TVP), an intra-aortic balloon pump (IABP), trans-aortic drainage catheters, left atrial decompression, pulmonary artery drainage cannulas, or placement of an arterial ECMO cannula in the thoracic aorta.

28. The method of any one of the previous paragraphs, wherein, in instances of treatment with a cardioprotective agent, the cardioprotective agent comprises a mitochondrial protective agent, an antioxidant, or an oxygen radical scavenger, wherein the oxygen radical scavenger is optionally selected from a nitroxide, such as Tempol (4-hydroxy-2,2,6,6-tetramethylpiperydine-1-oxyl) or Tiron (4,5-dihydroxy-1,3-benzenedisulfonic acid).

29. The method of paragraph 28, wherein the mitochondrial protective agent stabilizes cardiolipin, protects tafazzin from ECMO-induced depletion or reduction, or targets a protease that causes tafazzin degradation, wherein optionally a mitochondrial protease inhibitor is used, which optionally is a ClpP inhibitor (e.g., AV167, TG42, TG53, and TG54) or a ClpXP inhibitor.

30. The method of any one of the previous paragraphs, wherein, in instances of treatment with a cardioprotective agent, the cardioprotective agent comprises an aromatic tetrapeptide.

31. The method of any one of the previous paragraphs, wherein, in instances of treatment with a cardioprotective agent, the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

32. The method of any one of the previous paragraphs comprising LV decompression followed by ECMO treatment, wherein the LV decompression is commenced at least 10, 15, 20, 25, 30, 35, 40, 45, 50, 55, 60, 75, 90, 105, 120, 135, 150, 165, 180, 195, or 210 minutes before ECMO treatment begins.

33. The method of paragraph 32, wherein the LV decompression is carried out no longer than 90, 105, 120, 135, 150, 165, 180, 195, or 210 minutes before ECMO treatment is commenced.

34. The method of paragraph 32 or 33, wherein the LV decompression commences 10-180, 15-150, 20-120, 25-90, 15-45, 30-60, 45-75, 60-90, 75-105, 90-120, 105-135, 120-150, 135-165, 150-210, 105-195, or 120-180 minutes before commencement of ECMO treatment.

35. The method of any one of the previous paragraphs comprising LV decompression followed by ECMO treatment, wherein the flow rate of LV decompression is greater than the flow rate of ECMO.

36. The method of paragraph 35, wherein the flow rate for LV decompression is 3-5 L/minute, while the flow rate for ECMO is 2-4 L/minute, provided that the LV decompression flow rate is greater than the ECMO flow rate.

37. The method of paragraph 36, wherein the LV decompression flow rate is 3.5 to 4.5 L/minute, while ECMO flow rate is 3-4 L/minute, provided that the LV decompression flow rate is greater than the ECMO flow rate.

38. The method of paragraph 37, wherein the LV decompression flow rate is 3.5 L/minute, while the ECMO flow rate is 4 L/minute.

39. The method of any one of the previous paragraphs comprising LV decompression followed by ECMO treatment, wherein once ECMO has begun, combined LV decompression and ECMO treatment is carried out for at least 30, 45, 60, 75, 90, 105, 120, 135, 150, 165, 180, 195, or 210 minutes.

40. The method of paragraph 39, wherein the combined LV decompression and ECMO treatment is carried out for no more than 90, 105, 120, 135, 150, 165, 180, 195, or 210 minutes.

41. The method of paragraph 39 or 40, wherein the combined LV decompression and ECMO treatment is carried out for 30-60, 45-75, 60-90, 75-105, 90-120, 105-135, 120-150, 135-165, 150-180, 165-195, or 180-210 minutes.

42. A system comprising an ECMO machine and a cardioprotective agent or an ECMO machine and a LV decompression device.

43. The system of paragraph 42, wherein the cardioprotective agent comprises a mitochondrial protective agent, an antioxidant, or an oxygen radical scavenger.

44. The system of paragraph 42 or 43, wherein the mitochondrial protective agent stabilizes cardiolipin, protects tafazzin from ECMO-induced depletion or reduction, or targets a protease that causes tafazzin degradation.

45. The system of any one of paragraphs 42 to 44, wherein the cardioprotective agent comprises an aromatic tetrapeptide.

46. The system of any one of paragraphs 42 to 45, wherein the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

47. A composition for use in reducing or preventing myocardial damage in a subject caused by extracorporeal membrane oxygenation (ECMO), the composition comprising a cardioprotective agent.

48. The composition for use of paragraph 47, wherein the cardioprotective agent comprises a mitochondrial protective agent, an antioxidant, or an oxygen radical scavenger.

49. The composition for use of paragraph 48, wherein the mitochondrial protective agent stabilizes cardiolipin, protects tafazzin from ECMO-induced depletion or reduction, or targets a protease that causes tafazzin degradation.

50. The composition for use of any one of paragraphs 47 to 49, wherein the cardioprotective agent comprises an aromatic tetrapeptide.

51. The composition for use of any one of paragraphs 47 to 50, wherein the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

52. A kit comprising: (a) one or more disposable medical product for use with an ECMO machine, and (b) a cardioprotective agent.

53. The kit of paragraph 52, wherein the one or more disposable medical product is selected from the group consisting of a connector, a cannula, a tubing, or a filter.

54. The kit of paragraph 52 or 53, wherein the cardioprotective agent comprises a mitochondrial protective agent, an antioxidant, or an oxygen radical scavenger.

55. The kit of paragraph 54, wherein the mitochondrial protective agent stabilizes cardiolipin, protects tafazzin from ECMO-induced depletion or reduction, or targets a protease that causes tafazzin degradation.

56. The kit of any one of paragraphs 52 to 55, wherein the cardioprotective agent comprises an aromatic tetrapeptide.

57. The kit of any one of paragraphs 52 to 56, wherein the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are within the scope of the claims.

What is claimed is:

1. A method for reducing or preventing myocardial damage in a subject caused by extracorporeal membrane oxygenation (ECMO), the method comprising administering a cardioprotective agent to the subject and treating the subject with ECMO.

2. A method of reducing or preventing myocardial damage in a subject caused by ECMO, the method comprising treating the subject with normoxemic ECMO.

3. A method for reducing or preventing myocardial damage in a subject caused by ECMO, the method comprising treating the subject with left ventricular (LV) decompression prior to ECMO treatment.

4. The method of claim 1, wherein:
(i) the myocardial damage is caused by reduction or depletion of tafazzin levels in cardiac muscle cells of the subject, and the method restores or maintains said tafazzin levels;
(ii) the myocardial damage is caused by reduction or depletion of cardiolipin levels in cardiac muscle cells of the subject, and the method restores or maintains said cardiolipin levels;
(iii) the myocardial damage is caused by increased iAAA protease (e.g., YME1) activity in cardiac muscle cells of the subject, and the method decreases iAAA protease levels, in order to restore or maintain tafazzin levels; and/or
(iv) the mitochondrial protective agent stabilizes cardiolipin, protects tafazzin from ECMO-induced depletion or reduction, or targets a protease that causes tafazzin degradation.

5. A method of reducing, preventing, or treating myocardial damage in a subject caused by ECMO, the method comprising restoring or maintaining tafazzin levels in cardiac muscle cells of the subject by enzyme replacement, gene therapy, CRISPR, mRNA therapy, protease inhibitors, or siRNA, which is directed to tafazzin directly, cardiolipin, and/or an iAAA protease (e.g., YME1).

6. The method of claim 5, further comprising treating the subject with ECMO, optionally with preemptive LV decompression, which optionally is carried out prior to initiation of ECMO.

7. The method of claim 2, further comprising administering a cardioprotective agent to the subject.

8. The method of claim 1, wherein the cardioprotective agent is administered before, during, or after the ECMO treatment, or in any combination thereof.

9. The method of claim 2, wherein the subject is being treated for ischemic heart disease.

10. The method of claim 9, wherein the ischemic heart disease is selected from acute myocardial infarction, heart failure, shock, and high risk percutaneous coronary intervention (PCI).

11. The method of claim 2, further comprising treating the subject with coronary artery reperfusion.

12. The method of claim 11, wherein, in instances of treatment with a cardioprotective agent, the cardioprotective agent is administered before the start of coronary artery reperfusion.

13. The method of claim 12, wherein the cardioprotective agent is administered at least 30 minutes before the start of coronary artery reperfusion.

14. The method of claim 1, wherein the myocardial damage comprises a myocardial infarction or an increase in the size of an already existing myocardial infarction.

15. The method of claim 1, wherein the myocardial damage comprises left ventricular (LV) injury.

16. The method of claim 1, wherein the myocardial damage is characterized by oxidative stress.

17. The method of claim 11, wherein the myocardial damage is ischemia-reperfusion injury.

18. The method of claim 1, wherein the subject has or is at risk of developing myocardial infarction, heart failure, cardiac arrest, heart muscle disease, myocarditis, sepsis, hypothermia, post-transplant complications, cardiogenic shock, cardio-respiratory failure, respiratory failure, lung infection, acute respiratory distress syndrome, pulmonary embolism, congenital diaphragmatic hernia, influenza, pulmonary hypertension, pneumonia, respiratory failure, trauma, or Covid-19, or is subject to treatment with or by a ventricular assist device, heart transplant, lung transplant, heart surgery, or cardiac catheterization.

19. The method of claim 1, wherein the cardioprotective agent is administered by an intra-arterial, intra-coronary, intra-myocardial, intra-epicardial, pericardial, or intravenous route, or via the ECMO circuit.

20. The method of claim 1, further comprising intravenous administration of a cardioprotective agent.

21. The method of claim 11, wherein ECMO treatment is maintained during the coronary artery reperfusion.

22. The method of claim 1, wherein the ECMO is veno-arterial ECMO or veno-venous ECMO.

23. The method of claim 22, wherein the VA-ECMO is peripheral VA-ECMO.

24. The method of claim 1, wherein the ECMO is normoxemic ECMO.

25. The method of claim 1, further comprising LV decompression before initiation of ECMO.

26. The method of claim 3, wherein the LV decompression is carried out at least about 30 minutes before initiation of ECMO.

27. The method of claim 3, wherein the LV decompression is carried out using a trans-valvular pump (TVP), an intra-aortic balloon pump (IABP), trans-aortic drainage catheters, left atrial decompression, pulmonary artery drainage cannulas, or placement of an arterial ECMO cannula in the thoracic aorta.

28. The method of claim 1, wherein, in instances of treatment with a cardioprotective agent, the cardioprotective agent comprises a mitochondrial protective agent, an antioxidant, or an oxygen radical scavenger, wherein the oxygen radical scavenger is optionally selected from a nitroxide, such as Tempol (4-hydroxy-2,2,6,6-tetramethylpiperydine-1-oxyl) or Tiron (4,5-dihydroxy-1,3-benzenedisulfonic acid).

29. The method of claim 28, wherein the mitochondrial protective agent stabilizes cardiolipin, protects tafazzin from ECMO-induced depletion or reduction, or targets a protease that causes tafazzin degradation, wherein optionally a mitochondrial protease inhibitor is used, which optionally is a ClpP inhibitor (e.g., AV167, TG42, TG53, and TG54) or a ClpXP inhibitor.

30. The method of claim 1, wherein, in instances of treatment with a cardioprotective agent, the cardioprotective agent comprises an aromatic tetrapeptide.

31. The method of claim 1, wherein, in instances of treatment with a cardioprotective agent, the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

32. The method of claim 3, wherein the LV decompression is commenced at least 10, 15, 20, 25, 30, 35, 40, 45, 50, 55, 60, 75, 90, 105, 120, 135, 150, 165, 180, 195, or 210 minutes before ECMO treatment begins.

33. The method of claim 32, wherein the LV decompression is carried out no longer than 90, 105, 120, 135, 150, 165, 180, 195, or 210 minutes before ECMO treatment is commenced.

34. The method of claim 32, wherein the LV decompression commences 10-180, 15-150, 20-120, 25-90, 15-45, 30-60, 45-75, 60-90, 75-105, 90-120, 105-135, 120-150, 135-165, 150-210, 105-195, or 120-180 minutes before commencement of ECMO treatment.

35. The method of claim 3, wherein the flow rate of LV decompression is greater than the flow rate of ECMO.

36. The method of claim 35, wherein the flow rate for LV decompression is 3-5 L/minute, while the flow rate for ECMO is 2-4 L/minute, provided that the LV decompression flow rate is greater than the ECMO flow rate.

37. The method of claim 36, wherein the LV decompression flow rate is 3.5 to 4.5 L/minute, while ECMO flow rate is 3-4 L/minute, provided that the LV decompression flow rate is greater than the ECMO flow rate.

38. The method of claim 37, wherein the LV decompression flow rate is 3.5 L/minute, while the ECMO flow rate is 4 L/minute.

39. The method of claim 3, wherein once ECMO has begun, combined LV decompression and ECMO treatment is carried out for at least 30, 45, 60, 75, 90, 105, 120, 135, 150, 165, 180, 195, or 210 minutes.

40. The method of claim 39, wherein the combined LV decompression and ECMO treatment is carried out for no more than 90, 105, 120, 135, 150, 165, 180, 195, or 210 minutes.

41. The method of claim 39 er 40, wherein the combined LV decompression and ECMO treatment is carried out for 30-60, 45-75, 60-90, 75-105, 90-120, 105-135, 120-150, 135-165, 150-180, 165-195, or 180-210 minutes.

42. A system comprising an ECMO machine and a cardioprotective agent or an ECMO machine and a LV decompression device.

43. The system of claim 42, wherein the cardioprotective agent comprises a mitochondrial protective agent, an antioxidant, or an oxygen radical scavenger.

44. The system of claim 42, wherein the mitochondrial protective agent stabilizes cardiolipin, protects tafazzin from ECMO-induced depletion or reduction, or targets a protease that causes tafazzin degradation.

45. The system of claim 42, wherein the cardioprotective agent comprises an aromatic tetrapeptide.

46. The system of claim 42, wherein the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).

47-51. (canceled)

52. A kit comprising: (a) one or more disposable medical product for use with an ECMO machine, and (b) a cardioprotective agent.

53. The kit of claim 52, wherein the one or more disposable medical product is selected from the group consisting of a connector, a cannula, a tubing, or a filter.

54. The kit of claim 52, wherein the cardioprotective agent comprises a mitochondrial protective agent, an antioxidant, or an oxygen radical scavenger.

55. The kit of claim 54, wherein the mitochondrial protective agent stabilizes cardiolipin, protects tafazzin from ECMO-induced depletion or reduction, or targets a protease that causes tafazzin degradation.

56. The kit of claim 52, wherein the cardioprotective agent comprises an aromatic tetrapeptide.

57. The kit of claim 52, wherein the cardioprotective agent comprises (a) D-Arg-2′,6′-Dmt-Lys-Phe-NH₂ (MTP-131), (b) L-Phe-D-Arg-L-Phe-L-Lys-NH₂ (SBT-20), (c) a compound of a table herein (e.g., one or more of Tables 1-4 and A-E), or (d) a pharmaceutically acceptable salt or crystal form of any one of (a), (b), or (c).


SEQUENCE LISTING (41 sequences)

SEQ ID NO: 1 - protein, 4 aa, Artificial Sequence
  Synthetic Construct
  MISC_FEATURE (1)..(1): Xaa is 2′,6′-dimethylphenylalanine
  MISC_FEATURE (2)..(2): Xaa is D-Arg
  MISC_FEATURE (3)..(3): Xaa is 2′,6′-dimethyltyrosine
XXXK

SEQ ID NO: 2 - protein, 4 aa, Artificial Sequence
  Synthetic Construct
  MISC_FEATURE (1)..(1): Xaa is 2′,6′-dimethylphenylalanine
  MISC_FEATURE (2)..(2): Xaa is D-Arg
XXFK

SEQ ID NO: 3 - protein, 4 aa, Artificial Sequence
  Synthetic Construct
  MISC_FEATURE (1)..(1): Xaa is 2′,6′-dimethyltyrosine
  MISC_FEATURE (2)..(2): Xaa is D-Arg
  MISC_FEATURE (4)..(4): Xaa is Ornithine
XXFX

SEQ ID NO: 4 - protein, 4 aa, Artificial Sequence
  Synthetic Construct
  MISC_FEATURE (1)..(1): Xaa is 2′,6′-dimethyltyrosine
  MISC_FEATURE (2)..(2): Xaa is D-Arg
  MISC_FEATURE (4)..(4): Xaa is 2-amino-heptanoicacid
XXFX

SEQ ID NO: 5 - protein, 4 aa, Artificial Sequence
  Synthetic Construct
  MISC_FEATURE (1)..(1): Xaa is 2′,6′-dimethyltyrosine
  MISC_FEATURE (2)..(2): Xaa is D-Arg
XXFK

SEQ ID NO: 6 - protein, 4 aa, Artificial Sequence
  Synthetic Construct
  MISC_FEATURE (1)..(1): Xaa is 2′,6′-dimethyltyrosine
  MISC_FEATURE (2)..(2): Xaa is D-citrulline
XXFK

SEQ ID NO: 7 - protein, 10 aa, Artificial Sequence
  Synthetic Construct
  MISC_FEATURE (2)..(2): Xaa is D-Phe
  MISC_FEATURE (3)..(3): Xaa is D-Arg
  MISC_FEATURE (6)..(6): Xaa is D-Trp
  MISC_FEATURE (8)..(8): Xaa is D-Tyr
AXXYKXHXGF

SEQ ID NO: 8 - protein, 14 aa, Artificial Sequence
  Synthetic Construct
  MISC_FEATURE (2)..(2): Xaa is D-Leu
  MISC_FEATURE (3)..(3): Xaa is D-Tyr
  MISC_FEATURE (7)..(7): Xaa is D-Lys
  MISC_FEATURE (9)..(9): Xaa is D-Trp
  MISC_FEATURE (11)..(11): Xaa is D-Phe
  MISC_FEATURE (13)..(13): Xaa is D-Arg
RXXFKEXRXKXYXG

SEQ ID NO: 9 - protein, 16 aa, Artificial Sequence
  Synthetic Construct
  MISC_FEATURE (3)..(3): Xaa is D-Phe
  MISC_FEATURE (6)..(6): Xaa is D-Arg
  MISC_FEATURE (7)..(7): Xaa is D-Lys
  MISC_FEATURE (10)..(10): Xaa is D-Tyr
  MISC_FEATURE (12)..(12): Xaa is D-His
  MISC_FEATURE (14)..(14): Xaa is D-Phe
DRXCFXXYRXWXYXKF

SEQ ID NO: 10 - protein, 9 aa, Artificial Sequence
  Synthetic Construct
  MISC_FEATURE (2)..(2): Xaa is D-Trp
  MISC_FEATURE (5)..(5): Xaa is D-His
  MISC_FEATURE (8)..(8): Xaa is D-Gly
DXKYXFRXK

SEQ ID NO: 11 - protein, 4 aa, Artificial Sequence
  Synthetic Construct
  MISC_FEATURE (1)..(1): Xaa is D-Arg
  MISC_FEATURE (2)..(2): Xaa is 2′,6′-dimethyltyrosine
XXKF

SEQ ID NO: 12 - protein, 15 aa, Artificial Sequence
  Synthetic Construct
  MISC_FEATURE (1)..(1): Xaa is D-Glu
  MISC_FEATURE (4)..(4): Xaa is D-Arg
  MISC_FEATURE (5)..(5): Xaa is D-His
  MISC_FEATURE (8)..(8): Xaa is D-Val
  MISC_FEATURE (12)..(12): Xaa is D-Tyr
XDKXXFFXYRYXRHF

SEQ ID NO: 13 - protein, 6 aa, Artificial Sequence
  Synthetic Construct
  MISC_FEATURE (1)..(1): Xaa is D-His
  MISC_FEATURE (5)..(5): Xaa is D-Phe
XEKYXR

SEQ ID NO: 14 - protein, 11 aa, Artificial Sequence
  Synthetic Construct
  MISC_FEATURE (1)..(1): Xaa is D-His
  MISC_FEATURE (4)..(4): Xaa is D-Phe
  MISC_FEATURE (6)..(6): Xaa is D-Asp
  MISC_FEATURE (7)..(7): Xaa is D-Asp
  MISC_FEATURE (8)..(8): Xaa is D-His
  MISC_FEATURE (9)..(9): Xaa is D-Lys
XKYXEXXXXRW

SEQ ID NO: 15 - protein, 3 aa, Artificial Sequence
  Synthetic Construct
  MISC_FEATURE (1)..(1): Xaa is D-Tyr
XWK

SEQ ID NO: 16 - protein, 13 aa, Artificial Sequence
  Synthetic Construct
  MISC_FEATURE (3)..(3): Xaa is D-Lys
  MISC_FEATURE (5)..(5): Xaa is D-Val
  MISC_FEATURE (7)..(7): Xaa is D-His
  MISC_FEATURE (10)..(10): Xaa is D-Gly
  MISC_FEATURE (13)..(13): Xaa is D-Met
ERXYXFXWRXYRX

SEQ ID NO: 17 - protein, 19 aa, Artificial Sequence
  Synthetic Construct
  MISC_FEATURE (5)..(5): Xaa is D-Lys
  MISC_FEATURE (10)..(10): Xaa is D-Arg
  MISC_FEATURE (11)..(11): Xaa is D-Arg
  MISC_FEATURE (15)..(15): Xaa is D-His
  MISC_FEATURE (18)..(18): Xaa is D-Lys
GAKFXERYHXXDYWXWHXD

SEQ ID NO: 18 - protein, 6 aa, Artificial Sequence
  Synthetic Construct
  MISC_FEATURE (2)..(2): Xaa is D-Phe
  MISC_FEATURE (5)..(5): Xaa is D-Arg
GXKHXY

SEQ ID NO: 19 - protein, 17 aa, Artificial Sequence
  Synthetic Construct
  MISC_FEATURE (3)..(3): Xaa is D-Arg
  MISC_FEATURE (7)..(7): Xaa is D-Asp
  MISC_FEATURE (11)..(11): Xaa is D-Tyr
  MISC_FEATURE (14)..(14): Xaa is D-Lys
HYXWKFXARCXHFXYHS

SEQ ID NO: 20 - protein, 3 aa, Artificial Sequence
  Synthetic Construct
  MISC_FEATURE (2)..(2): Xaa is D-Arg
KXY

SEQ ID NO: 21 - protein, 6 aa, Artificial Sequence
  Synthetic Construct
  MISC_FEATURE (2)..(2): Xaa is D-Gln
  MISC_FEATURE (5)..(5): Xaa is D-Phe
KXYRXW

SEQ ID NO: 22 - protein, 8 aa, Artificial Sequence
  Synthetic Construct
  MISC_FEATURE (3)..(3): Xaa is D-Tyr
  MISC_FEATURE (8)..(8): Xaa is D-His
KWXRNFYX

SEQ ID NO: 23 - protein, 5 aa, Artificial Sequence
  Synthetic Construct
  MISC_FEATURE (3)..(3): Xaa is D-Arg
MYXFR

SEQ ID NO: 24 - protein, 5 aa, Artificial Sequence
  Synthetic Construct
  MISC_FEATURE (3)..(3): Xaa is D-Lys
MYXFR

SEQ ID NO: 25 - protein, 4 aa, Artificial Sequence
  Synthetic Construct
  MISC_FEATURE (3)..(3): Xaa is D-His
FRXD

SEQ ID NO: 26 - protein, 4 aa, Artificial Sequence
  Synthetic Construct
  MISC_FEATURE (2)..(2): Xaa is D-Arg
  MISC_FEATURE (3)..(3): Xaa is 2′,6′-dimethyltyrosine
FXXK

SEQ ID NO: 27 - protein, 3 aa, Artificial Sequence
  Synthetic Construct
  MISC_FEATURE (2)..(2): Xaa is D-Arg
FXH

SEQ ID NO: 28 - protein, 7 aa, Artificial Sequence
  Synthetic Construct
  MISC_FEATURE (2)..(2): Xaa is D-Arg
  MISC_FEATURE (6)..(6): Xaa is D-Arg
FXKWYXH

SEQ ID NO: 29 - protein, 4 aa, Artificial Sequence
  Synthetic Construct
  MISC_FEATURE (2)..(2): Xaa is D-Arg
FXFK

SEQ ID NO: 30 - protein, 11 aa, Artificial Sequence
  Synthetic Construct
  MISC_FEATURE (3)..(3): Xaa is D-Tyr
  MISC_FEATURE (7)..(7): Xaa is D-Lys
  MISC_FEATURE (9)..(9): Xaa is D-Arg
FFXREDXRXHF

SEQ ID NO: 31 - protein, 12 aa, Artificial Sequence
  Synthetic Construct
  MISC_FEATURE (4)..(4): Xaa is D-Arg
  MISC_FEATURE (7)..(7): Xaa is D-Lys
  MISC_FEATURE (8)..(8): Xaa is D-Lys
  MISC_FEATURE (11)..(11): Xaa is D-Tyr
FYKXWHXXERXT

SEQ ID NO: 32 - protein, 9 aa, Artificial Sequence
  Synthetic Construct
  MISC_FEATURE (5)..(5): Xaa is D-His
  MISC_FEATURE (8)..(8): Xaa is D-His
TGYRXFWXK

SEQ ID NO: 33 - protein, 20 aa, Artificial Sequence
  Synthetic Construct
  MISC_FEATURE (4)..(4): Xaa is D-Lys
  MISC_FEATURE (9)..(9): Xaa is D-Lys
  MISC_FEATURE (10)..(10): Xaa is D-Arg
  MISC_FEATURE (13)..(13): Xaa is D-Tyr
  MISC_FEATURE (17)..(17): Xaa is D-His
TYRXWYEDXXHFXGVIXRYK

SEQ ID NO: 34 - protein, 4 aa, Artificial Sequence
  Synthetic Construct
  MISC_FEATURE (2)..(2): Xaa is D-Lys
WXYR

SEQ ID NO: 35 - protein, 8 aa, Artificial Sequence
  Synthetic Construct
  MISC_FEATURE (4)..(4): Xaa is D-Asp
  MISC_FEATURE (7)..(7): Xaa is D-His
WKFXRYXK

SEQ ID NO: 36 - protein, 12 aa, Artificial Sequence
  Synthetic Construct
  MISC_FEATURE (3)..(3): Xaa is D-Lys
  MISC_FEATURE (6)..(6): Xaa is D-Lys
  MISC_FEATURE (7)..(7): Xaa is D-Arg
  MISC_FEATURE (10)..(10): Xaa is D-Tyr
YDXYFXXFPXHK

SEQ ID NO: 37 - protein, 5 aa, Artificial Sequence
  Synthetic Construct
  MISC_FEATURE (2)..(2): Xaa is D-Arg
YXFKE

SEQ ID NO: 38 - protein, 4 aa, Artificial Sequence
  Synthetic Construct
  MISC_FEATURE (2)..(2): Xaa is D-Arg
YXFK

SEQ ID NO: 39 - protein, 11 aa, Artificial Sequence
  Synthetic Construct
  MISC_FEATURE (2)..(2): Xaa is D-His
  MISC_FEATURE (4)..(4): Xaa is D-Arg
  MISC_FEATURE (7)..(7): Xaa is D-Arg
  MISC_FEATURE (10)..(10): Xaa is D-His
YXFXDKXHWXF

SEQ ID NO: 40 - protein, 4 aa, Artificial Sequence
  Synthetic Construct
  MISC_FEATURE (3)..(3): Xaa is D-Gly
YHXM

SEQ ID NO: 41 - protein, 8 aa, Artificial Sequence
  Synthetic Construct
  MISC_FEATURE (2)..(2): Xaa is D-Lys
  MISC_FEATURE (5)..(5): Xaa is D-Phe
VXHYXSYR